(19) United States
(12) Patent Application Publication
(10) Pub. No.: US 2016/0195533 A1
(43) Pub. Date: Jul. 7, 2016

(54) METHOD FOR SCREENING EMT INHIBITOR

(71) Applicant: Medicinal Bioconvergence Research Center, Suwon-si (KR)

(72) Inventors: Byung-Gyu KIM, Daegu (KR); Ji-Ae Song, Suwon-si (KR); Sunghoon Kim, Seoul (KR)

(21) Appl. No.: 14/670,390

(22) Filed: Mar. 26, 2015

(30) Foreign Application Priority Data
Jan. 2, 2015 (KR) 10-2015-0000234

(51) Int. Cl. G01N 33/573

(52) U.S. Cl. CPC G01N 33/573; G01N 2500/02; G01N 2333/9015

(57) ABSTRACT

A method for screening an EMT inhibitor including: contacting EPRS, Snail1 protein, and a test agent; and measuring a change in a binding level between the EPRS and the Snail1 protein.

### CROSS-REFERENCE TO RELATED APPLICATION

This application claims priority from and the benefit of Korean Patent Application No. 10-2015-0000234, filed on Jan. 2, 2015, which is hereby incorporated by reference for all purposes as if fully set forth herein.

### BACKGROUND

1. Field

Exemplary embodiments relate to a method for screening an epithelial-mesenchymal transition (EMT) inhibitor.

2. Discussion of the Background

Epithelial-mesenchymal transition (EMT), which occurs during a normal embryonic development procedure, is a process by which epithelial cells lose their epithelial cell phenotype and obtain the mesenchymal cell phenotype with high mobility. However, it has been known that irreversible EMT does not only cause heart, liver, kidney, and vascular malfunctions, but is also involved in the transition to malignancy. When EMT occurs, epithelial cells lose their apical-basal polarity, change their shape from a square type to a fibroblast type, have a reduced number of epithelial cell markers, and an increased number of mesenchymal cell markers. It has been recently known that EMT plays various roles in the regeneration and fibrosis of tissue and the development and transition of cancer as well as embryonic histogenesis and differentiation.

Snail is currently known to be involved in the EMT procedure by inhibiting E-cadherin, which is an invasion inhibitor, in the process of cancer metastasis, and is known to be also associated with prenatal mesoderm and neutral tube formation. Experimental results known today proved that Snail is involved in melanoma, bladder cancer, rectal cancer, pancreatic cancer, and the like (see Cano, A. et al. Nat Cell Biol 2, 76-83 (2000); Batlle, E. et al. Nat Cell Biol 2, 84-9 (2000); Poser, I. et al. J Biol Chem 276, 24661-6 (2001); and De Craene, B. et al. Cancer Res 65, 6237-44 (2005)). It is known that significant reduction in the expression level of E-cadherin in such cancers was associated with the overexpression of the Snail protein. The expression of the Snail protein is regulated in TGF and Wnt signaling pathways on the transcription level.

TGF-β is one of the main growth factors in wound healing (summarized in O'Kane (1997) Int J Biochem Cell Biol 29:79-89). During granulation, TGF-β comes from platelets in the wounded area. Here, TGF-β regulates its generation in macrophages, and induces secretion of other growth factors by, for example, monocytes. The important functions thereof during wound healing include promotion of chemotaxis of inflammatory cells, synthesis of extracellular matrix, and regulation of gene expression, proliferation, and differentiation of cell types, which are involved in the wounding healing process. Under pathological conditions, these TGF-β-mediated effects, especially, the regulation of the extracellular matrix (ECM) generation may cause fibrosis or intradermal wounding (Border (1994) N Engl J Med 331:1286-1292).

It has been reported that TGF-β promotes renal cell hypertrophy and pathogenic accumulation of the extracellular matrix in fibrotic disease, diabetic nephropathy, and glomerulonephritis. The interruption of the TGF-β signaling pathway through treatment using an anti-TGF-β antibody prevents mesangial matrix expansion and gradual reduction of renal function, and reduces established lesions of diabetic glomerular disease in animals (see Border (1990) 346: 371-374; Yu (2004) Kindney Int 66: 1774-1784; Fukasawah (2004) Kindney Int 65: 63-74; and Sharma (1996) Diabetes 45: 522-530). TGF-β also plays an important role in liver fibrosis. The activation, essential for the development of liver fibrosis, of the hepatic stellate cells to give myofibroblasts, the main producer of the extracellular matrix in the course of the development of liver cirrhosis, is stimulated by TGF-β. It has likewise been shown here that interruption of the TGF-β signaling pathway reduces fibrosis in experimental models (see Yata (2002) Hepatology 35:1022-1030; and Arias (2003) BMC Gastroenterol 3:29).

TGF-β also takes on a key function in the formation of cancer (summarized in Derynck (2001) Nature Genetics: 29: 117-129; and Elliott (2005) J Clin Onc 23: 2078-2093). In early stages of the development of cancer, TGF-β counters the formation of cancer. This tumor-suppressive activity is based principally on the ability of TGF-β to inhibit the division of epithelial cells. By contrast, TGF-β promotes cancer growth and the formation of metastases in later tumor stages. This may result from the fact that most epithelial tumors develop resistance to the growth-inhibiting action of TGF-β, and TGF-β simultaneously supports the growth of cancer cells through other mechanisms. These mechanisms include the promotion of angiogenesis, immunosuppressive action, which supports tumor cells in avoiding the control function of the immune system (immune-surveillance), and promotion of invasiveness and the formation of metastases. The formation of the invasive phenotype of tumor cells is a principal prerequisite for the formation of metastases. TGF-β promotes this process through its ability to regulate cellular adhesion, motility, and the formation of the extracellular matrix. Furthermore, TGF-β induces the transition from the cell epithelial phenotype to the invasive mesenchymal phenotype (epithelial mesenchymal transition=EMT). The important role played by TGF-β in the promotion of cancer growth is also demonstrated by investigations which show a correlation between strong TGF-β expression and a poor prognosis. The increased TGF-β level has been found, inter alia, in patients with prostate, breast, intestinal, and lung cancer (see Wikstroem (1998) Prostate 37: 19-29; Hasegawa (2001) Cancer 91: 964-971; and Friedman (1995), Cancer Epidemiol Biomarkers Prev. 4:549-54).

Aminoacyl-tRNA synthetase is an enzyme that accurately binds a specific amino acid to its corresponding tRNA, and is essential in the protein synthesis system. The procedure of binding amino acid and tRNA is largely divided into two steps: a first step of activating the amino acid into aminoacyl adenylate by consuming one ATP; and a second step of transferring the activated tRNA. A mammalian aminoacyl tRNA synthetase has a role similar to that of a prokaryotic aminoacyl tRNA synthetase, but has a different additional domain. This additional domain is involved in the formation of various complexes with aminoacyl tRNA synthetase or other regulatory factors. It has recently been discovered that this structural complexity is associated with functional variety of the aminoacyl tRNA synthetase and several human diseases.

The above information disclosed in this Background section is only for enhancement of understanding of the background of the inventive concept, and, therefore, it may contain information that does not form the prior art that is already known in this country to a person of ordinary skill in the art.

### SUMMARY

One or more exemplary embodiments relate to a method for screening an epithelial-mesenchymal transition (EMT) inhibitor.

Additional aspects will be set forth in the detailed description which follows, and, in part, will be apparent from the disclosure, or may be learned by practice of the inventive concept.

One or more exemplary embodiments provide a method for screening an EMT inhibitor, the method including: (a) contacting glutamyl-prolyl-tRNA synthetase (EPRS) and Snail1 protein with a test agent; and (b) measuring a change in a binding level between the EPRS and the Snail1 protein. The amino acid sequence may be selected from the group consisting of SEQ ID NOs: 2 to 6.

One or more exemplary embodiments provide a method for screening an epithelial-mesenchymal transition (EMT) inhibitor, the method including: (a) contacting glutamyl-prolyl-tRNA synthetase (EPRS) having an amino acid sequence, Snail1 protein, and a test agent, the amino acid sequence being selected from the group consisting of SEQ ID NOS: 2 to 6; and (b) measuring a change in a binding level between the EPRS and the Snail1 protein.

One or more exemplary embodiments relate to a novel use of EPRS, and provide a method for screening an EMT inhibitor. The teachings of this disclosure may be useful to develop novel therapeutic agents against various EMT-related diseases, including canner.

The foregoing general description and the following detailed description are exemplary and explanatory and are intended to provide further explanation of the claimed subject matter.

### BRIEF DESCRIPTION OF THE DRAWINGS

The accompanying drawings, which are included to provide a further understanding of the inventive concept, and are incorporated in and constitute a part of this specification, illustrate exemplary embodiments of the inventive concept, and, together with the description, serve to explain principles of the inventive concept.

In all drawings herein, the sign + expresses the presence of a corresponding material (i.e., expression by transfection and material treatment condition), and the sign − expresses the absence of the corresponding material.

FIG. 1 shows western blot analysis results, after A549 cells were treated with TGF-β1 dose-dependently, and then nucleus and cytosol fractions were subjected to endogenous immunoprecipitation (IP) using protein A-agarose beads and anti-EPRS antibody), according to one or more exemplary embodiments of the inventive concept. (WB/SNAIL: western blotting with anti-Snail 1 antibody, WB/EPRS: western blotting with anti-EPRS antibody, Cyto: cytosol fraction, Nuc: nucleus fraction).

FIG. 2 shows immunoblot assay of EPRS and Snail1), according to one or more exemplary embodiments of the inventive concept. HEK293T cells transiently co-transfected with Flag-tagged EPRS and Strep-tagged Snail1. EPRS was imunoprecipitated with anti-Flag M2 affinity gel and immunoblotted with anti-Strep-HRP and anti-Flag-HRP antibody.

FIG. 3 shows immunoblot assay of EPRS and so on. HEK293T cells transiently transfected with Strep-tagged Snail1), according to one or more exemplary embodiments of the inventive concept. Snail1 was imunoprecipitated with MagStrep 2HC and immunoblotted with anti-EPRS, anti-HDAC1, anti-L13a, anti-RRS, anti-Strep-HRP antibody. (STREP-EV: experimental group using HEK293T cell transfected with Strep-tagged empty vector, WCL: whole cell lysate).

FIG. 4 shows western blot analysis results, according to one or more exemplary embodiments of the inventive concept. In HEK293T cells in which Strep-tagged ERS-WHEP (indicated by E), Strep-tagged WHEP-PRS (indicated by P), or 2× Strep-tagged EPRS (indicated by EP) was expressed together with Flag-tagged Snail 1, immunoprecipitation (IP) using MagStrep 2HC was performed and western blotting was performed using anti-Flag-HRP antibody and anti-strep-HRP antibody (WCL: whole cell lysate).

FIG. 5A shows immunoblotting results, according to one or more exemplary embodiments of the inventive concept. Flag-tagged empty vector or Flag-tagged EPRS was expressed in A549 cells, which were then treated with or without TGF-β1, and then the cell lysate for each experimental group was immunoblotted with anti-Snail1 antibody, anti-SMAD2 antibody, anti-SMAD3 antibody, anti-FLAG-HRP antibody, and anti-β-actin antibody.

FIG. 5B shows immunoblotting results, according to one or more exemplary embodiments of the inventive concept. 2× strep-tagged empty vector or 2× strep-tagged EPRS was expressed in HCC44 cells, which were then treated with or without TGF-β1, and then the cell lysate for each experimental group was immunoblotted with anti-Snail 1 antibody and anti-strep-HRP antibody.

FIG. 5C shows immunoblotting results, according to one or more exemplary embodiments of the inventive concept. 2× strep-tagged empty vector or 2× strep-tagged EPRS was expressed in HEK293T cells, which were then treated with or without TGF-β1, and then the cell lysate for each experimental group was immunoblotted with anti-Snail 1 antibody and anti-strep-HRP antibody.

FIG. 5D shows immunoblotting results, according to one or more exemplary embodiments of the inventive concept. 2× strep-tagged empty vector or 2× strep-tagged EPRS was expressed at different expression levels in H1299 cells, and then the cell lysate for each experimental group was immunoblotted with anti-Snail 1 antibody and anti-strep-HRP antibody (MOCK: empty vector, [symbol] : the expression level of a corresponding protein increases in cells)

FIG. 6 shows results when A549 cells transiently transfected for overexpression of 2×Strep-tagged EPRS were treated with or without TGF-β1, and then subjected to qRT-PCR assay to monitor the mRA expression level of Snail (A) and EPRS (B), according to one or more exemplary embodiments of the inventive concept. (MocK: experimental group using A549 cells transfected with 2×Strep-tagged empty vector plasmid DNA, EPRS: experimental group using A549 cells transfected with 2×Strep-tagged EPRS plasmid DNA, Cont: TGF-β1 non-treated group, TGFβ1: TGF-β1 treated group).

FIG. 7 shows immunoblotting results, according to one or more exemplary embodiments of the inventive concept. Each of Strep-tagged ERS, Strep-tagged PRS, and Strep-tagged EPRS, and Flag-tagged Snail 1 were alone or together in HEK293T cells, and then the cell lysates in the MG 132 treated group and MG 132 untreated group were immunoblotted with anti-Flag-HRP antibody (EV: STREP-tag empty vector and FLAG-tag empty vector expression).

FIG. 8. Shows the effect of EPRS on ubiqutination of Snail1, according to one or more exemplary embodiments of the inventive concept. HA-tagged ubiquitin was co-transfected with Strep-Snail and Flag-Mock or Flag-EPRS in the presence of 50 μM MG132 for 4 h before harvest. The cell lysates were immunoprecipitated with streptavidin agarose (GE healthcare lifesciences). The beads were washed three times with the cold washing buffer, the precipitates were dissolved in the 2×SDS sample buffer and subjected to SDS-PAGE. Subsequently, Western blot analysis was performed with anti-HA antibody. (WCL: whole cell lysate).

FIG. 9A shows immunoblotting results, according to one or more exemplary embodiments of the inventive concept. EPRS-knocked-down A549 cells were treated with or without TGF-β1 for 1 hour, and the cell lysates were fractionated into nucleus and cytoplasmic protein. Then, EMT marker proteins of Snail 1, p-SMAD3, SMAD2/3, SLUG, and EPRS were immunoblotted (si-con: non-silencing control siRNA introduced group; si-EPRS: human EPRS gene-specific siRNA introduced knockdown group; HSP90α/β and p84 were used as loading controls for confirming the protein expression levels in cytoplasm and nucleus).

FIG. 9B shows immunoblotting results, according to one or more exemplary embodiments of the inventive concept. EPRS-knocked-down A549 cells were treated with or without TGF-β1 for 48 hours, and the cell lysate was fractionated into the nucleus and cytoplasmic protein. Then, EMT marker proteins of E-cadherin, N-cadherin, and EPRS were immunoblotted (si-con: non-silencing control siRNA introduced group; si-EPRS: human EPRS gene-specific siRNA introduced knockdown group; HSP90α/β and p84 were used as loading controls for confirming the protein expression amounts in cytoplasm and nucleus).

FIG. 10 shows microscope magnified images of cell migration assay results in groups of EPRS-knocked-down A549 cells treated with or without TGF-β1, according to one or more exemplary embodiments of the inventive concept. (si-con: non-silencing control siRNA introduced group; si-EPRS: human EPRS-specific siRNA introduced knockdown group).

FIG. 11 shows quantifications of cell migration assay results in groups of EPRS-knocked-down A549 cells treated with or without TGF-β1, according to one or more exemplary embodiments of the inventive concept. (si-con: non-silencing control siRNA introduced group; si-EPRS: human EPRS-specific siRNA introduced knockdown group).

FIG. 12 shows microscope magnified images of cell invasion assay results in groups of EPRS-knocked-down A549 cells treated with or without TGF-β1, according to one or more exemplary embodiments of the inventive concept. (si-con: non-silencing control siRNA introduced group; si-EPRS: human EPRS-specific siRNA introduced knockdown group).

FIG. 13 shows quantifications of cell invasion assay results in groups of EPRS-knocked-down A549 cells treated with or without TGF-β1, according to one or more exemplary embodiments of the inventive concept. (si-con: non-silencing control siRNA introduced group; si-EPRS: human EPRS-specific siRNA introduced knockdown group).

FIG. 14 is a diagram showing domains constituting each of polypeptides represented by SEQ ID NOS: 1 to 7, according to one or more exemplary embodiments of the inventive concept. (for each, “EPRS (WT)” corresponds SEQ ID NO: 1, “EPRS(ΔPRS)” corresponds SEQ ID NO: 2, “EPRS(ΔWHEP, PRS)” corresponds SEQ ID NO: 3, “EPRS(ΔERS)” corresponds SEQ ID NO: 4, “EPRS(ΔERS, WHEP)” corresponds SEQ ID NO: 5, “EPRS(ΔWHEP)” corresponds SEQ ID NO: 6, and “EPRS(ΔERS, PRS)” corresponds SEQ ID NO: 7).

### DETAILED DESCRIPTION OF THE ILLUSTRATED EMBODIMENTS

In the following description, for the purposes of explanation, numerous specific details are set forth in order to provide a thorough understanding of various exemplary embodiments. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments.

In the accompanying figures, the size and relative sizes of layers, films, panels, regions, etc., may be exaggerated for clarity and descriptive purposes. Also, like reference numerals denote like elements.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

The present inventors, while searching for methods and materials for effectively treating various epithelial-mesenchymal transition (EMT)-related diseases, such as cancer and fibrosis, have found a novel function of EPRS that EMT is activated by the physical interaction between EPRS and Snail1, and verified that the inhibition of the binding of EPRS and Snail1 suppresses EMT. One or more exemplary embodiments described herein are based upon the research and discovery noted above.

Therefore, exemplary embodiments will be provided in view of the above-mentioned problems, and one or more exemplary embodiments provide a method for screening an epithelial-mesenchymal transition (EMT) inhibitor, the method including: (a) contacting full-length glutamyl-prolyl-tRNA synthetase (EPRS) or its fragment having the amino acid sequence selected from the group consisting of SEQ ID NOS: 2 to 6, Snail1 protein, and a test agent; and (b) measuring the change in the binding level between EPRS and Snail1.

Hereinafter, various examples and embodiments will be described in detail.

The present inventors discovered that EPRS interacts with Snail1 to regulate an EMT pathway, and the details of which will be also discussed.

### DEFINITIONS

Unless defined otherwise, all technical and scientific terms used herein have the same meaning as commonly understood by a person skilled in the art. The following reference documents provide one of skills having general definitions with many terms used herein: Singleton et al., DICTIONARY OF MICROBIOLOGY AND MOLECULAR BIOLOGY(2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY. Also, the following definitions are provided to help readers with the implementation of the inventive concept.

As used herein, the term “protein” is used interchangeably with the term “polypeptide” or “peptide”, and refers to a polymer of amino acid residues, as typically found in proteins in nature.

As used herein, the term “expression” refers to the formation of protein or nucleic acid in cells.

As used herein, the term “glutamyl-prolyl-tRNA synthetase (EPRS)” refers to a type of aminoacyl-tRNA synthetase that promotes the binding of glutamate and proline with tRNA. Among aminoacyl-tRNA synthetases, the enzyme (EPRS) that promotes the binding of glutamate and proline with tRNA in humans is uniquely present on one polypeptide chain. A glutamate-catalyzing domain and a proline-catalyzing domain are located in the N-terminal region and the C-terminal region, respectively, and the two domains are linked via the WHEP domain. The glutamyl tRNA synthetase pertains to class 1 aminoacyl tRNA synthetase, and the proline tRNA synthetase pertains to class 2 aminoacyl tRNA synthetase. The WHEP domain linking the two domains has a structure in which the chain of 57 amino acids is repeated three times, and mediates a protein-protein interaction or a protein-RNA interaction. As long as the full-length EPRS protein (or polypeptide) of exemplary embodiments is any known mammal-derived EPRS sequence, the kind thereof is not particularly limited, and for example, it may be a human EPRS protein represented by SEQ ID NO: 1. In addition, the EPRS of the protein may encompass its functional equivalents.

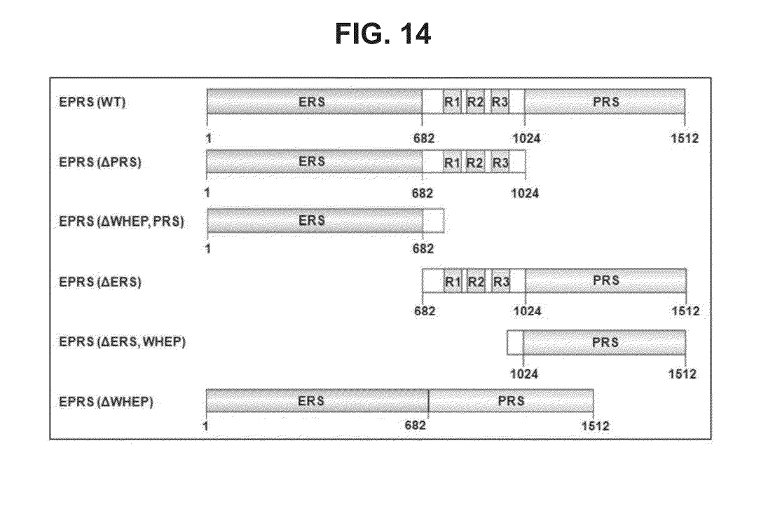

As used herein, the term “EPRS fragment” refers to a sequence of a fragment from the full-length EPRS polypeptide, and may be preferably any sequence that contains a glutamate binding domain (ERS domain) or/and a proline binding domain (PRS domain) without particular limitation, and include, for example, SEQ ID NO: 2 (ERS-WHEP domain, “EPRS (ΔPRS) in FIG. 14”, that is, extracted from the sequence of 1^{st} to 1024^{th} amino acids from the EPRS sequence represented by SEQ ID NO: 1), SEQ ID NO: 3 (ERS domain, “EPRS(ΔWHEP, PRS) in FIG. 14”, that is, extracted from the sequence of 1^{st} to 682^{nd} amino acids from the EPRS sequence represented by SEQ ID NO: 1), SEQ ID NO: 4 (WHEP-PRS domain, “EPRS(ΔERS) in FIG. 14”, that is, extracted from the sequence of 682^{nd} to 1512^{th} amino acids from the EPRS sequence represented by SEQ ID NO: 1), SEQ ID NO: 5 (PRS domain, “EPRS (ΔERS, WHEP) in FIG. 14”, that is, extracted from the sequence of 1024^{th} to 1512^{th} amino acids from the EPRS sequence represented by SEQ ID NO: 1), and SEQ ID NO: 6 (ERS-PRS domain, a linked body of SEQ ID NO: 3 and SEQ ID NO: 5). Herein, the EPRS fragment may be, most preferably, one that contains a proline binding domain, and may be, for example, any one polypeptide selected from the group consisting of SEQ ID NO: 4, SEQ ID NO: 5, and SEQ ID NO: 6, but is not limited thereto. Further, the EPRS fragment of exemplary embodiments may include its functional equivalents.

The term “functional equivalent” refers to a polypeptide having sequence homology (that is, identity) of at least 70%, preferably 80% or more, and more preferably 90% or more to the amino acid sequence of EPRS or its fragment. For example, the functional equivalent encompasses polypeptides having sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%, and refers to a polypeptide exhibiting substantially identical physiological activity as the polypeptide represented by any one selected from the group consisting of SEQ ID NO: 1 to SEQ ID NO: 6. Herein, the term “substantially identical physiological activity” refers to the activity to provoke and promote the progress of the EMT pathway through binding with Snail, particularly, Snail1. The functional equivalent may result from the addition, substitution, or deletion of a part of the amino acid sequence of EPRS or its fragments. Herein, the substitution of amino acid is preferably a conservative substitution. Examples of the naturally occurring amino acid conservative substitution are as follows: aliphatic amino acids (Gly, Ala, Pro), hydrophobic amino acids (Ile, Leu, Val), aromatic amino acids (Phe, Tyr, Trp), acidic amino acids (Asp, Glu), basic amino acids (His, Lys, Arg, Gln, Asn), and sulfur-containing amino acids (Cys, Met). In addition, the functional equivalent encompasses variants in which some amino acids are deleted from the amino acid sequence of the EPRS polypeptide. The deletion or substitution of the amino acids is preferably located in a region which is not directly involved in physiological activity of the EPRS polypeptide. The functional equivalent also encompasses variants in which some amino acids are added to both terminals of the amino acid sequence of the EPRS polypeptide or into the amino acid sequence of the EPRS polypeptide. In addition, the functional equivalent also encompasses a polypeptide derivative in which a basic frame of the polypeptide according to one or more exemplary embodiments and physiological activity thereof are maintained, and the chemical structure of the polypeptide is modified. For example, the functional equivalent also encompasses the structural change for changing stability, storage ability, volatility, or solubility of the polypeptide of exemplary embodiments.

Herein, the sequence homology and identity are defined as the percentage of amino acid residues of a candidate sequence over the EPRS amino acid sequence after the amino acid sequence of the EPRS amino acid sequence (SEQ ID NO: 1) or its fragments (SEQ ID NO: 2 to SEQ ID NO: 6) and the candidate sequence are aligned and then gaps are introduced. If necessary, in order to obtain the sequence identity with the maximum percentage, the conservative substitution as a part of the sequence identity is not considered. In addition, none of N-terminal, C-terminal, or internal extensions, deletions, or insertions of the amino acid sequence of EPRS or its fragments shall be construed as affecting sequence identity or homology. In addition, the sequence homology may be determined by a general standard method used to compare similar parts of amino acid sequences of two polypeptides. A computer program, such as BLAST, aligns two polypeptides such that amino acids thereof are optimally matched (either along the full length of one or both sequences or along a predicted portion of one or both sequences). The program provides a default opening penalty and a default gap penalty, and provides a scoring matrix that can be used in connection with the computer program, for example, PAM250 (standard scoring matrix; Dayhoff et al., in Atlas of Protein Sequence and Structure, vol 5, supp 3, 1978). For example, the percent identity may be calculated as follows. The total number of identical matches multiplied by 100 and then divided by the sum of the length of the longer sequence within the matched span and the number of gaps introduced into the longer sequences in order to align the two sequences.

Herein, preferable functional equivalents of the EPRS encompass: NCBI Gene Bank Accession Number EAW93309.1 (SEQ ID NO: 25), CAI45949.1 (SEQ ID NO: 26), XP_001172425.1 (SEQ ID NO: 27), XP_003807230.1 (SEQ ID NO: 28), XP_009439782.1 (SEQ ID NO: 29), XP_003265132.1 (SEQ ID NO: 30), EAW93310.1 (SEQ ID NO: 31), and CAA38224.1 (SEQ ID NO: 32), which are 99% identical to the amino acid sequence of SEQ ID NO: 1; NCBI Gene Bank Accession Number XP_010366377.1 (SEQ ID NO: 33), XP_005540939.1 (SEQ ID NO: 34), XP_007986597.1 (SEQ ID NO: 35), XP_005540938.1 (SEQ ID NO: 36), XP_007986596.1 (SEQ ID NO: 37), XP_007986598.1 (SEQ ID NO: 38), which are 98% identical to the amino acid sequence of SEQ ID NO: 1; NCBI Gene Bank Accession Number XP_003930387.1 (SEQ ID NO: 39), XP_010339237.1 (SEQ ID NO: 40), EHHSO530.1 (SEQ ID NO: 41), which are 97% identical to the amino acid sequence of SEQ ID NO: 1; NCBI Gene Bank Accession Number XP_002808297.1 (SEQ ID NO: 42), XP_009185375.1 (SEQ ID NO: 43), EHH15538.1 (SEQ ID NO: 44), which are 96% identical to the amino acid sequence of SEQ ID NO: 1; NCBI Gene Bank Accession Number XP_004028474.1 (SEQ ID NO: 45), XP_008983438.1 (SEQ ID NO: 46), which are 95% identical to the amino acid sequence of SEQ ID NO: 1; NCBI Gene Bank Accession Number XP_008053500.1 (SEQ ID NO: 47), which are 94% identical to the amino acid sequence of SEQ ID NO: 1; NCBI Gene Bank Accession Number XP_001488980.1 (SEQ ID NO: 48), XP_008541056.1 (SEQ ID NO: 49), XP_849468.1 (SEQ ID NO: 50), XP_004013652.1 (SEQ ID NO: 51), XP_005640882.1 (SEQ ID NO: 52), XP_005981363.1 (SEQ ID NO: 53), XP_005690536.1 (SEQ ID NO: 54), XP_002920053.1 (SEQ ID NO: 55), XP_005981362.1 (SEQ ID NO: 56), XP_004271072.1 (SEQ ID NO: 57), XP_008053499.1 (SEQ ID NO: 58), XP_004415195.1 (SEQ ID NO: 59), XP_004324147.1 (SEQ ID NO: 60), EFB20321.1 (SEQ ID NO: 61), XP_004751515.1 (SEQ ID NO: 62), XP_008683957.1 (SEQ ID NO: 63), XP_003999559.1 (SEQ ID NO: 64), XP_007072912.1 (SEQ ID NO: 65), XP_008586040.1 (SEQ ID NO: 66), which are 93% identical to the amino acid sequence of SEQ ID NO: 1; NCBI Gene Bank Accession Number XP_006188215.1 (SEQ ID NO: 67), XP_006198933.1 (SEQ ID NO: 68), XP_004480290.1 (SEQ ID NO: 69), XP_008266633.1 (SEQ ID NO: 70), XP_006056008.1 (SEQ ID NO: 71), XP_007935118.1 (SEQ ID NO:72), XP_007455449.1 (SEQ ID NO: 73), NP_001230249.1 (SEQ ID NO: 74), XP_006056007.1 (SEQ ID NO: 75), XP_005905625.1 (SEQ ID NO: 76), XP_007129327.1 (SEQ ID NO: 77), XP_007172136.1 (SEQ ID NO: 78), XP_005335922.1 (SEQ ID NO: 79), XP_007172135.1 (SEQ ID NO: 80), which are 92% identical to the amino acid sequence of SEQ ID NO: 1; NCBI Gene Bank Accession Number XP_003419927.1 (SEQ ID NO: 81), XP_006869760.1 (SEQ ID NO: 82), XP_007521381.1 (SEQ ID NO: 83), XP_005335921.1 (SEQ ID NO: 84), XP_008148043.1 (SEQ ID NO: 85), XP_004439743.1 (SEQ ID NO: 86), XP_004685497.1 (SEQ ID NO: 87), XP_004578688.1 (SEQ ID NO: 88), XP_005879946.1 (SEQ ID NO: 89), which are 91% identical to the amino acid sequence of SEQ ID NO: 1; NCBI Gene Bank Accession Number XP_008825021.1 (SEQ ID NO: 90), XP_008825020.1 (SEQ ID NO: 91), XP_004699962.1 (SEQ ID NO: 92), XP_006140298.1 (SEQ ID NO: 93), XP_007642504.1 (SEQ ID NO: 94), ERE73005.1 (SEQ ID NO: 95), AAH94679.1 (SEQ ID NO: 96), EDL13067.1 (SEQ ID NO: 97), XP_008825022.1 (SEQ ID NO: 98), which are 90% identical to the amino acid sequence of SEQ ID NO: 1; NCBI Gene Bank Accession Number XP_005348894.1 (SEQ ID NO: 99), AAI41050.1 (SEQ ID NO: 100), NP_084011.1 (SEQ ID NO: 101), XP_006497180.1 (SEQ ID NO: 102), XP_005082493.1 (SEQ ID NO: 103), ERE73006.1 (SEQ ID NO: 104), XP_007636777.1 (SEQ ID NO: 105), which are 89% identical to the amino acid sequence of SEQ ID NO: 1; NCBI Gene Bank Accession Number XP_004670165.1 (SEQ ID NO: 106), XP_006916292.1 (SEQ ID NO: 107), XP_006250488.1 (SEQ ID NO: 108), XP_006250487.1 (SEQ ID NO: 109), XP_006094389.1 (SEQ ID NO: 110), which are 88% identical to the amino acid sequence of SEQ ID NO: 1; NCBI Gene Bank Accession Number XP_005409334.1 (SEQ ID NO: 111), XP_004626936.1 (SEQ ID NO: 112), XP_004613070.1 (SEQ ID NO: 113), KFO25396.1 (SEQ ID NO: 114), which are 87% identical to the amino acid sequence of SEQ ID NO: 1; NCBI Gene Bank Accession Number XP_003474570.1 (SEQ ID NO: 115), XP_005005650.1 (SEQ ID NO: 116), XP_004867452.1 (SEQ ID NO: 117), XP_004878984.1 (SEQ ID NO: 118), which are 86% identical to the amino acid sequence of SEQ ID NO: 1; NCBI Gene Bank Accession Number EPQ16188.1 (SEQ ID NO: 119), which is 85% identical to the amino acid sequence of SEQ ID NO: 1; NCBI Gene Bank Accession Number NP_001019409.1 (SEQ ID NO: 120), which is 84% identical to the amino acid sequence of SEQ ID NO: 1; and NCBI Gene Bank Accession Number XP_008177290.1 (SEQ ID NO: 121), XP_009094640.1 (SEQ ID NO: 122), XP_006022406.1 (SEQ ID NO: 123), which are 83% identical to the amino acid sequence of SEQ ID NO: 1, but not limited thereto.

The protein (polypeptide) according to one or more exemplary embodiments may be naturally extracted or may be constructed by a genetic engineering method. For example, a nucleic acid encoding the polypeptide or its functional equivalent (SEQ ID NO: 8 for EPRS, and any one selected from the group consisting of SEQ ID NOS: 9 to 13 for the EPRS fragment) is constructed by a normal method. The nucleic acid may be constructed by PCR amplification using appropriate primers. Alternatively, the DNA sequence may be synthesized by a standard method known in the art, for example, an automatic DNA synthesizer (marketed by Biosearch Co. or Applied Biosystems Co.) The constructed nucleic acid is inserted into a vector including at least one expression control sequence (e.g., promoter, enhancer, etc.), which is operatively linked to the nucleic acid to regulate the expression of the nucleic acid, and the resulting recombinant expression vector transfects host cells. The resultant transformants are cultured under media and conditions suitable for the expression of the nucleic acid, and a substantially pure polypeptide which is expressed by the nucleic acid is collected from the cultured product. The collection may be conducted using the method known in the art (e.g., chromatography). Herein, the term “substantially pure polypeptide” means that the polypeptide according to one or more exemplary embodiments does not substantially contain any other protein derived from host cells. The genetic engineering method for synthesizing the polypeptide of the present invention may refer to the following documents: Maniatis et al., Molecular Cloning; A laboratory Manual, Cold Spring Harbor laboratory, 1982; Sambrook et al., Molecular Cloning: A Laboratory Manual, Cold Spring Harbor Press, N.Y., Second (1998) and Third (2000) Editions; Gene Expression Technology, Method in Enzymology, Genetics and Molecular Biology, Method in Enzymology, Guthrie & Fink(eds.), Academic Press, San Diego, Calif., 1991; and Hitzeman et al., J. Biol. Chem., 255:12073-12080, 1990.

In addition, the polypeptide of one or more exemplary embodiments may be easily prepared by a chemical synthesis known in the art (Creighton, Proteins; Structures and Molecular Principles, W. H. Freeman and Co., NY, 1983). Representative examples thereof include, but are not limited to, liquid- or solid-phase synthesis, fragment condensation, F-MOC or T-BOC chemical synthesis (Chemical Approaches to the Synthesis of Peptides and Proteins, Williams et al., Eds., CRC Press, Boca Raton Fla., 1997; A Practical Approach, Athert on & Sheppard, Eds., IRL Press, Oxford, England, 1989).

As used herein, the term “promoter” refers to a DNA sequence that regulates expression of a nucleic acid sequence operatively linked thereto in the particular host cell, and the term “operably linked” means that one nucleic acid fragment binds to other nucleic acid fragments so that the function or expression of one is affected by the other. In addition, the promoter may further include any operator sequence for regulating transcription, a sequence coding an appropriate mRNA ribosome binding site, and a sequence regulating the termination of transcription and translation. The promoter may use a constitute promoter that ordinarily induces the expression of a target gene in all time zones, or an inducible promoter that induces the expression of a target gene at a particular location and time.

As used herein, the term “host cells” refers to prokaryotic or eukaryotic cells including heterologous DNA that is newly introduced into cells by any means (e.g., electric shock, calcium phosphatase precipitation, microinjection, transfection, viral infection, etc.)

As used herein, the term “nucleic acid”. “DNA sequence”, or “polynucleotide” refers to a single- or double-stranded deoxyribonucleotide or ribonucleotide. Unless otherwise limited, the term encompasses known analogs of natural nucleotides that hybridize to nucleic acids in a manner similar to naturally-occurring nucleotides.

As used herein, the term “polynucleotide encoding EPRS” may have a nucleotide sequence encoding an amino acid sequence represented by SEQ ID NO: 1 or an amino acid sequence having sequence homology of at least 70% to the foregoing amino acid sequence. The nucleic acid includes all DNA, cDNA, and RNA sequences. Specifically, the polynucleotide may have a nucleotide sequence encoding an amino acid sequence of SEQ ID NO: 1 or an amino acid sequence encoding having homology of at least 70% to the foregoing amino acid sequence, or may have a nucleotide sequence complementary to the foregoing nucleotide sequence. Preferably, the polynucleotide may have a nucleotide sequence represented by SEQ ID NO: 8. The nucleic acid may be isolated from nature, or may be constructed by the genetic engineering method described as above.

In addition, the polynucleotide sequence encoding the EPRS fragment may have a nucleotide sequence encoding an amino acid sequence selected from the group consisting of SEQ ID NOS: 2 to 6 or an amino acid sequence having sequence homology of at least 70% to the foregoing amino acid sequence. Preferably, the polynucleotide sequence may have any one nucleotide sequence selected from the group consisting of SEQ ID NOS: 9 to 13. The nucleic acid may be isolated from nature, or may be constructed by the genetic engineering method described as above.

As used herein, the term “homologues” when referring to proteins and/or protein sequences indicates that they are derived, naturally or artificially, from a common ancestral protein or protein sequence. Similarly, nucleic acids and/or nucleotide sequences are homologous when they are derived, naturally or artificially, from a common ancestral nucleic acid or nucleic acid sequence.

As used herein, the term “analog” refers to a molecule that is structurally similar to a reference molecule but has been modified in view of a target and a regulatory manner by replacing a specific substituent of the reference molecule with an alternate substituent. Compared to the reference molecule, the analog has similar or improved utility, which would be expected by a person skilled in the art. Synthesis and screening of analogs for identifying variants of known compounds having improved features (e.g., higher binding affinity to a target molecule) are well known in the pharmaceutical chemistry field.

As used herein, the term “contacting” has a general meaning, and refers to binding two or more agents (e.g., two polypeptides) or binding an agent and cells (e.g., protein and cells). Contacting may occur in vitro. For example, two or more agents are bound or a test agent and cells or a test agent and a cell lysate are bound in a test tube or another container. In addition, contacting may occur in cells or in situ. For example, recombinant polynucleotides encoding two polypeptides are co-expressed in cells, so that two polypeptides are contacted with each other in the cells or cell lysate.

As used herein, the term “agent” or “test agent” encompasses any substance, molecule, element, compound, entity, or a combination thereof. For example, the term encompasses, but is not limited to, protein, polypeptide, small organic molecule, polysaccharide, polynucleotide, and the like. Moreover, the term may be a natural product, a synthetic compound, a chemical compound, or a combination of two or more substances. Unless otherwise specified, the terms “agent”, “substance” and “compound” can be used interchangeably.

More specifically, the test agent that can be screened by the screening method of one or more exemplary embodiments includes polypeptides, β-turn mimetics, polysaccharides, phospholipids, hormones, prostaglandins, steroids, aromatic compounds, heterocyclic compounds, benzodiazepines, oligomeric N-substituted glycines, oligocarbamates, saccharides, fatty acids, purine, pyrimidine or derivatives, structural analogs, or a combinations thereof. A certain test agent may be a synthetic substance, and another test agent may be a natural substance. The test agent may be obtained from a wide variety of sources including synthetic or natural compound libraries. A combinatorial library may be produced from several kinds of compounds that can be synthesized in a step-by-step manner. Compounds of multiple combinatorial libraries may be prepared by the encoded synthetic libraries (ESL) method (WO 95/12608, WO 93/06121, WO 94/08051, WO 95/395503, and WO 95/30642). A peptide library may be generated by a phage display method (WO 91/18980). Libraries of natural compounds of bacteria, mold, plant, and animal extracts may be obtained from commercial sources or collected from fields. The known pharmacological agents may be applied to directed or random chemical modifications, such as acylation, alkylation, esterification, and amidification, in order to prepare structural analogs.

The test agent may be a naturally occurring protein or a fragment thereof. This test agent may be obtained from a natural source, for example, a cell or a tissue lysate. A library of polypeptide agents may also be obtained, for example, from a cDNA library, which is created by established routine methods or commercially available. The test agents may be peptides, such as peptides having about 5 to about 30 amino acids, preferably about 5 to about 20 amino acids, and more preferably from about 7 to about 15 amino acids. The peptides may represent the degraded products of naturally occurring proteins, random peptides, or “biased” random peptides.

Alternatively, the test agent may be a “nucleic acid”. The nucleic acid test agent may be a naturally occurring nucleic acid, random nucleic acid, or “biased” random nucleic acid. For example, the degraded products of prokaryotic or eukaryotic genomes can be used in a similar way as described above.

In addition, the test agents may be small molecules (e.g., molecules with a molecular weight of not more than about 1000). Preferably, high throughput assay may be applied for screening small molecule modulators. Many assays are useful for the screening (Shultz, Bioorg. Med. Chem. Lett., 8:2409-2414, 1998; Weller, Mol. Drivers., 3:61-70, 1997; Fernandes, Curr. Opin. Chem. Biol., 2:597-603, 1998; and Sittampalam, Curr. Opin. Chem. Biol., 1:384-91, 1997).

Libraries of test agents to be screened according to the method of one or more exemplary embodiments may be created on the basis of structural studies of Snail1 and EPRS, or fragments or analogs thereof. Such structural studies allow the identification of test agents that are more likely bind to Snail1 or EPRS. The three-dimensional structure of Snail1 or EPRS may be explored in a number of ways, such as crystal structure and molecular modeling. Protein structure studying methods using X-ray crystallography are well known in the document: Physical Bio-Chemistry, Van Holde, K. E. (Prentice-Hall, New Jersey 1971), pp. 221-239, and Physical Chemistry with Applications to the Life Sciences, D. Eisengerg & D. Crothers (Benjamin Cummings, Menlo Park 1979). Computer modeling for Snail1 or EPRS structures is provided as another means for designing test agents for screening. Molecular modeling methods are disclosed in the documents: U.S. Pat. No. 612,894, and U.S. Pat. No. 5,583,973. Further, protein structures may also be determined using neutron diffraction and nuclear magnetic resonance (NMR). Physical Chemistry, 4th Ed. Moore, W. J. (Prentice-Hall, New Jersey 1972) and NMR of Proteins and Nucleic Acids, K. Wuthrich (Wiley-Interscience, New York 1986).

One or more exemplary embodiments provide a method for screening an epithelial-mesenchymal transition (EMT) inhibitor.

The method includes (a) contacting full-length glutamyl-prolyl-tRNA synthetase (EPRS) or its fragment having the amino acid sequence selected from the group consisting of SEQ ID NOS: 2 to 6, Snail1 protein, and a test agent; and (b) measuring the change in the binding level between EPRS and Snail1.

As used herein, the term “Snail1” encompasses human Snail1 protein or homologous Snail1 protein of non-human origin (preferably mammalian origin) having equivalent functions to the human Snail1 protein. For example, the term may be the human Snail1 protein represented by SEQ ID NO: 15, but is not limited thereto. Further, in one or more exemplary embodiments, the Snail1 encompasses its functional equivalents.

The polynucleotide encoding Snail1 may have a nucleotide sequence encoding an amino acid sequence represented by SEQ ID NO: 15 or an amino acid sequence having sequence homology of at least 70% to the foregoing amino acid sequence. Preferably, it may have a nucleotide sequence represented by SEQ ID NO: 16. The nucleic acid may be isolated from the nature, or may be constructed by the genetic engineering method described as above.

The functional equivalent of Snail1 refers to a polypeptide having sequence homology (that is, identity) of at least 70%, preferably 80% or more, and more preferably 90% or more to the amino acid sequence of Snail1. Herein, specific functional equivalents of Snail1 encompass: NCBI Gene Bank Accession Number AAD17332.1 (SEQ ID NO: 124), BAG36039.1 (SEQ ID NO: 125), XP_004062397.1 (SEQ ID NO: 126), AAF32527.1 (SEQ ID NO: 127), which have 99% sequence identity to the Snail1 amino acid sequence of SEQ ID NO: 1; NCBI Gene Bank Accession Number XP_003809303.1 (SEQ ID NO: 128), XP_003252972.1 (SEQ ID NO: 129), XP_010382148.1 (SEQ ID NO: 130), XP_001097698.1 (SEQ ID NO: 131), XP_002830458.1 (SEQ ID NO: 132), XP_009435687.1 (SEQ ID NO: 133), which have 98% sequence identity to the Snail1 amino acid sequence of SEQ ID NO: 1; NCBI Gene Bank Accession Number XP_003932616.1 (SEQ ID NO: 134), which has 97% sequence identity to the Snail1 amino acid sequence of SEQ ID NO: 1; NCBI Gene Bank Accession Number XP_003787739.1 (SEQ ID NO: 135), which have 95% sequence identity to the Snail1 amino acid sequence of SEQ ID NO: 1; NCBI Gene Bank Accession Number XP_008060134.1 (SEQ ID NO: 136), which has 93% sequence identity to the Snail1 amino acid sequence of SEQ ID NO: 1; NCBI Gene Bank Accession Number XP_008570069.1 (SEQ ID NO: 137), XP_004883645.1 (SEQ ID NO: 138), XP_003732892.1 (SEQ ID NO: 139), which have 92% sequence identity to the Snail1 amino acid sequence of SEQ ID NO: 1; NCBI Gene Bank Accession Number KF035879.1 (SEQ ID NO: 140), which has 91% sequence identity to the Snail1 amino acid sequence of SEQ ID NO: 1; NCBI Gene Bank Accession Number XP_003467769.1 (SEQ ID NO: 141), XP_006141336.1 (SEQ ID NO: 142), XP_005325275.1 (SEQ ID NO: 143), ELW71708.1 (SEQ ID NO: 144), EFB26680.1 (SEQ ID NO: 145), XP_008696422.1 (SEQ ID NO: 146), which have 90% sequence identity to the Snail1 amino acid sequence of SEQ ID NO: 1; NCBI Gene Bank Accession Number NP_446257.1 (SEQ ID NO: 147), XP_005392380.1 (SEQ ID NO: 148), XP_008833387.1 (SEQ ID NO: 149), XP_005362911.1 (SEQ ID NO: 150), XP_006992689.1 (SEQ ID NO: 151), XP_005688790.1 (SEQ ID NO: 152), XP_005635224.1 (SEQ ID NO: 153), XP_008532217.1 (SEQ ID NO: 154), which have 89% sequence identity to the Snail1 amino acid sequence of SEQ ID NO: 1; NCBI Gene Bank Accession Number XP_002913027.1 (SEQ ID NO: 155), XP_004687529.1 (SEQ ID NO: 156), XP_004412574.1 (SEQ ID NO: 157), XP_007185370.1 (SEQ ID NO: 158), XP_007446447.1 (SEQ ID NO: 159), XP_007128099.1 (SEQ ID NO: 160), XP_004282941.1 (SEQ ID NO: 161), NP_035557.1 (SEQ ID NO: 162), XP_004746271.1 (SEQ ID NO: 163), XP_006206723.1 (SEQ ID NO: 164), XP_006175044.1 (SEQ ID NO: 165), NP_001106179.1 (SEQ ID NO: 166), XP_004430338.1 (SEQ ID NO: 167), XP_005074485.1 (SEQ ID NO: 168), XP_007608553.1 (SEQ ID NO: 169), which have 88% sequence identity to the Snail1 amino acid sequence of SEQ ID NO: 1; NCBI Gene Bank Accession Number XP_005983433.1 (SEQ ID NO: 170), CAA47675.1 (SEQ ID NO: 171), XP_003983440.1 (SEQ ID NO: 172), XP_001501267.2 (SEQ ID NO: 173), XP_003502520.1 (SEQ ID NO: 174), XP_004014930.1 (SEQ ID NO: 175), which have 87% sequence identity to the Snail1 amino acid sequence of SEQ ID NO: 1; NCBI Gene Bank Accession Number XP_004663926.1 (SEQ ID NO: 176), XP_006758858.1 (SEQ ID NO: 177), XP_006098041.1 (SEQ ID NO: 178), XP_007936620.1 (SEQ ID NO: 179), XP_004462255.1 (SEQ ID NO: 180), XP_008139391.1 (SEQ ID NO: 181), XP_004636165.1 (SEQ ID NO: 182), XP_006921969.1 (SEQ ID NO: 183), XP_004370603.1 (SEQ ID NO: 184), XP_004618816.1 (SEQ ID NO: 185), XP_004325726.1 (SEQ ID NO: 186), which have 86% sequence identity to the Snail1 amino acid sequence of SEQ ID NO: 1; NCBI Gene Bank Accession Number XP_007533425.1 (SEQ ID NO: 187), XP_003419974.1 (SEQ ID NO: 188), XP_005574198.1 (SEQ ID NO: 189), AAQ21376.1 (SEQ ID NO: 190), XP_007641286.1 (SEQ ID NO: 191), which have 85% sequence identity to the Snail1 amino acid sequence of SEQ ID NO: 1; NCBI Gene Bank Accession Number XP_005866258.1 (SEQ ID NO: 192), XP_006839389.1 (SEQ ID NO: 193), XP_004698375.1 (SEQ ID NO: 194), XP_006881815.1 (SEQ ID NO: 195), EHH21632.1 (SEQ ID NO: 196), which have 84% sequence identity to the Snail1 amino acid sequence of SEQ ID NO: 1; NCBI Gene Bank Accession Number XP_005860983.1 (SEQ ID NO: 197), which has 83% sequence identity to the Snail1 amino acid sequence of SEQ ID NO: 1; NCBI Gene Bank Accession Number XP_005658936.1 (SEQ ID NO: 198), which has 81% sequence identity to the Snail1 amino acid sequence of SEQ ID NO: 1; NCBI Gene Bank Accession Number XP_006099783.1 (SEQ ID NO: 199), EAW70471.1 (SEQ ID NO: 200), XP_004585952.1 (SEQ ID NO: 201), which have 79% sequence identity to the Snail1 amino acid sequence of SEQ ID NO: 1; NCBI Gene Bank Accession Number XP_004033198.1 (SEQ ID NO: 202), which has 78% sequence identity to the Snail1 amino acid sequence of SEQ ID NO: 1; and NCBI Gene Bank Accession Number XP_004451686.1 (SEQ ID NO: 203), which have 77% sequence identity to the Snail1 amino acid sequence of SEQ ID NO: 1, but are not limited thereto.

As used herein, the term “epithelial-mesenchymal transition (EMT)” means transition of epithelial cells into mesenchymal cells and diseases associated therewith. It was found that EMT is relevant with many diseases, such as tissue fibrosis and cancer development. For example, it has been known that cells, especially, cancer cells obtain mobility through EMT as an initial procedure, and penetrate into surrounding tissue. This suggests that the suppression of the EMT process allows the prevention and treatment of many EMT-related diseases, such as tissue fibrosis and cancers, in the initial stage.

As used herein, the term “EMT inhibitor” refers to an agent for preventing and/or treating known EMT-inducible diseases or symptoms, and more specifically, may be an agent for preventing or treating diseases or symptoms selected from the group consisting of, specifically, cancer metastasis, fibrotic disease, angiogenesis, diabetic renal nephropathy, allograft dysfunction, cataracts, and defects in cardiac valve formation, but is not limited thereto.

Specifically, the cancer in the “cancer metastasis” may be selected from the group consisting of, for example, non-small cell lung cancer, small cell lung cancer, melanoma, leukemia, colon cancer, liver cancer, gastric cancer, esophageal cancer, pancreatic cancer, gallbladder cancer, kidney cancer, bladder cancer, prostate cancer, testicular cancer, cervical cancer, endometrial cancer, choriocarcinoma, ovarian cancer, breast cancer, thyroid cancer, brain cancer, head and neck cancer, skin cancer, lymphoma, aplastic anemia, bile duct cancer, oral cancer, peritoneal cancer, small intestine cancer, and eye tumor, but is not limited thereto.

In addition, the fibrosis may be selected from the group consisting of, for example, renal fibrosis, hepatic fibrosis, pulmonary fibrosis, skin fibrosis, cardiac fibrosis, joint fibrosis, nerve fibrosis, muscular fibrosis, and peritoneal fibrosis, but is not limited thereto.

The EMT inhibitor of one or more exemplary embodiments may be an agent for preventing or treating, preferably, cancer (especially, cancer metastasis), and may be an agent for preventing or treating, most preferably, non-small cell lung cancer (especially, metastasis of non-small cell lung cancer).

The term “binding” may be a direct or indirect binding of Snail1 protein and full-length EPRS or its fragment having any one amino acid sequence selected from the group consisting of SEQ ID NOS: 2 to 6. The indirect binding means that the binding between two proteins forms a complex via another factor or together with the factor.

Herein, the change in the binding level between EPRS and Snail1 may be preferably a reduction in the binding level.

The reduction in the binding level means removal, prevention, or suppression of the binding between EPRS and Snail1. Specifically, the reduction in the binding level may be performed by allowing a test agent to remove, prevent the generation of, or suppress the generation of EPRS or Snail1 to change the expression level of EPRS or Snail1, or may be conducted by a method by which a test agent competitively or non-competitively binds to EPRS or Snail1 to change the level of interaction (binding) between the two proteins, or a method by which the interaction (binding) between EPRS and Snail1 of the EPRS-Snail1 protein complex that has been previously formed in cells is removed by a test agent. The term “competitively bind” means that a test agent binds to a site at which EPRS and Snail1 interact with (bind to) each other to remove, prevent, or suppress the interaction between EPRS and Snail1, and the term “non-competitively bind” means that a test agent binds to one other than the site at which EPRS and Snail1 interact with (bind to) each other, to remove, prevent, or suppress the interaction between EPRS and Snail1. According to one or more exemplary embodiments, it screens a test agent which inhibits expression and inherent functions of EPRS or Snail1 and suppresses or reduces the intercellular interaction (binding) level between Snail1 and EPRS (in an independent manner) within the extent that it does not cause a side effect and inhibits.

Preferably, the reduction in the binding level of one or more exemplary embodiments may be preferably attained by a method by which a test agent comparatively or non-comparatively binds to EPRS or Snail1 to change the interaction (binding) level between the two, or a method by which the interaction (binding) between EPRS and Snail1 of the EPRS-Snail1 protein complex that has been previously formed in cells is removed by a test agent.

The test agents need not essentially functionally inhibit expression and inherent functions of EPRS or Snail1, and merely inhibiting the interaction (binding) between EPRS and Snail1 is enough.

According to one or more exemplary embodiments, the screening may be performed by various methods known in the art, such as protein-protein binding assay in a labeled test tube (in vitro full-down assay), EMSA, immunoassay for protein binding, functional assay (phosphorylation assay, etc.), yeast-2 hybrid assay, non-immunoprecipitation assay, immunoprecipitation western blot assay, immuno-co-localization assay, and the like, but are not limited thereto.

For example, yeast-2 hybrid assay may be carried out by using yeast expressing EPRS and Snail1, or parts or homologues of these proteins, fused with the DNA-binding domain of bacteria repressor LexA or yeast GAL4 and the transactivation domain of yeast GAL4 protein, respectively (KIM, M. J. et al., Nat. Gent., 34:330-336, 2003). The interaction between EPRS and Snail1 reconstructs a transactivator that induces the expression of a reporter gene under the control of a promoter having a regulatory sequence binding to the DNA-binding domain of LexA or GAL4 protein.

As described above, the reporter gene may be any gene that is known in the art and encodes a detectable polypeptide. For example, chloramphenicol acetyl transferase (CAT), luciferase, β-galactosidase, β-glucosidase, alkaline phosphatase, green fluorescent protein (GFP), or the like may be used. If the binding between EPRS and Snail1, or parts or homologues of these proteins is degraded or weakened by a test agent, the reporter gene is not expressed, or expressed less than under a normal condition.

Further, as the reporter gene, one that encodes a protein enabling the growth of yeast (i.e., the growth of yeast is inhibited if the reporter gene is not expressed) may be selected. For example, auxotropic genes that encode enzymes involved in biosynthesis for obtaining amino acids or nitrogen bases (e.g., yeast genes, such as ADE3 and HIS3, or similar genes from other species) may be used. In cases where the binding of EPRS and Snail1, or parts or homologues of these proteins, expressed in this system, is inhibited by a test agent, the reporter gene is not expressed. Accordingly, the growth of yeast is stopped or retarded under such a condition. Such an effect by the expression of the reporter gene may be observed with the naked eye or by using a device (e.g., a microscope).

After steps (a) and (b), step (c) below may be further carried out: (c) contacting the test agent, which has changed the binding level between EPRS and Snail1 in step (b), with cells expressing Snail1, together with TGF-β1, and then verifying the EMT inhibitory effect in the cells.

TGF-β1 is known to induce the transition from the cell epithelial phenotype to the invasive mesenchymal phenotype, that is, EMT. Snail1 is known to be relevant to the EMT phenotype, and the abundance (expression level) of Snail1 increases by TGF-β1 treatment (example 1). Therefore, in step (c), an additional process of treating any cell expressing Snail1 (especially, normal epithelial cells or the like) with TGF-β1 to induce EMT, and then verifying whether the test agent primarily selected through step (b) actually has a therapeutic effect on the EMT symptom.

In one or more exemplary embodiments, EPRS interacts with (binds to) Snail1 to provoke other EMT-related diseases, such as cancer and fibrosis, through the EMT pathway, and thus the test gene, which has changed the binding level between EPRS and Snail1 in step (b), can inhibit EMT.

As long as the cells expressing Snail1 are any known Snail1-expressing cells, the kind thereof is not particularly limited, but the cells expressing Snail1 may be epithelial cells, tumor cells, and fibrosis cells. The epithelial cells (normal epithelial cells) refer to cells that cover surfaces of the animal body or inner surface of blank spaces or tracts (internal organ tracts or the like), and the sites from which cells are derived, such as retina, colon, small intestine, blood vessels, skin, and the like, are not particularly limited.

Examples of cancer cells may be cells selected from the group consisting of non-small cell lung cancer cells, small cell lung cancer cells, melanoma cells, leukemia cells, colon cancer cells, liver cancer cells, gastric cancer cells, esophageal cancer cells, pancreatic cancer cells, gallbladder cancer cells, kidney cancer cells, bladder cancer cells, prostate cancer cells, testicular cancer cells, cervical cancer cells, endometrial cancer cells, choriocarcinoma cells, ovarian cancer cells, breast cancer cells, thyroid cancer cells, brain cancer cells, head and neck cancer cells, skin cancer cells, lymphoma cells, aplastic anemia cells, bile duct cancer cells, oral cancer cells, peritoneal cancer cells, small intestine cancer cells, and eye tumor cancer cells, or may be cells derived or differentiated therefrom, but are not limited thereto.

Examples of the fibrosis cells may be cells selected from the group consisting of renal fibrosis cells, hepatic fibrosis cells, pulmonary fibrosis cells, skin fibrosis cells, cardiac fibrosis cells, joint fibrosis cells, nerve fibrosis cells, muscular fibrosis cells, and peritoneal fibrosis cells, or may be cells derived or differentiated therefrom, but are not limited thereto.

The term “verifying the EMT inhibitory effect” refers to verifying whether a test agent inhibits the EMT progress provoked by TGF-β1 treatment in cells, and may be conducted through a comparison with, as a control group, Snail1-expressing cells treated with TGF-β1 but without the test agent. The method for verifying the EMT inhibitory effect may be conducted by confirming expression levels of known EMT markers, or confirming whether cell phenotypes exhibited by EMT, such as cell migration and cell invasion, are shown by known methods (e.g., cell migration assay, cell invasion assay), but is not limited thereto.

The term “EMT marker” refers to a protein that exhibits the expression which is specifically changed during the EMT, when compared with the normal state. As long as the EMT maker is any known EMT marker, the kind thereof is not particularly limited, but preferably, it may be one that can confirm the change in the expression of at least one marker protein selected from the group consisting of p-SMAD3, SMAD2/3, SLUG, E-cadherin, and N-cadherin.

The screening method of one or more exemplary embodiments may be conducted by using various biochemical and molecular biological techniques known in the art. The techniques are disclosed in the documents: Sambrook et al., Molecular Cloning: A Laboratory Manual, Cold Spring Harbor Press, N.Y., Second (1998) and Third (2000) Editions; and Ausubel et al., Current Protocols in Molecular Biology, John Wiley & Sons, Inc., New York (1987-1999).

Preferably, prior to the screening method including steps (a) and (b), or (a) to (c), the test agent may be first selectively assayed for its ability to modulate biological activity of EPRS or Snail1 (first assay stage). Specifically, in the first assay stage, the biological activity of EPRS or Snail1 isolated in the presence of the test gene is assayed to identify a modulating agent that modulates biological activity of the polypeptide. More preferably, exemplary embodiments may include the following steps: (i) contacting test agents with EPRS or Snail1 in the presence of the test agents; (ii) measuring activity of EPRS or Snail1 to select a test agent that changes the activity thereof.

In the first assay stage, the modulations of several biological activities of EPRS or Snail1 may be assayed. For example, a test agent may be assayed for its activity to modulate the expression level, e.g., transcription or translation. The test agent may also be assayed for its activity to modulate the intercellular level or stability, e.g., post-translational modification or proteolysis.

The modulating agent that changes biological activity is identified by the first assay stage, and then the test agent is tested for whether it has the ability to change the interaction between EPRS and Snail1, by the screening method of one or more exemplary embodiments including steps (a) and (b) or (a) to (c) (a second assay stage).

In both of the first and second assay stages, intact EPRS, and its fragments, analogs, or functional equivalents may be used. The fragments that can be used in the assays generally retain at least one biological activity of EPRS. In addition, fusion proteins including the fragments or analogs may be used to screen test agents. The functional equivalents of EPRS retain the same biological activities as EPRS although they have amino deletion and/or insertion and/or substitution, and thus can be used in the screening method of one or more exemplary embodiments, which is described above.

Various assays that are conventionally implemented in the art may be employed to identify EPRS or Snail1, or test agents that regulate the interaction therebetween. Preferably, the test agents may be screened by a cell-based assay system. For example, in a typical cell based assay for screening (i.e., the second test stage), the activity of the reporter gene (e.g., enzymatic activity) is measured in the presence of a test agent, and compared to the activity of the reporter gene in the absence of the test agent. The reporter gene may encode any detectable polypeptide (response or reporter polypeptide) known in the art, e.g., a polypeptide that is detectable by fluorescence or phosphorescence or by the enzymatic activity retained therein. The detectable response polypeptide may be, e.g., luciferase, alpha-glucuronidase, alpha-galactosidase, chloramphenicol acetyl transferase, green fluorescent protein, enhanced green fluorescent protein, and human secreted alkaline phosphatase.

In the cell-based assay, the test agent (e.g., a peptide or a polypeptide) may also be expressed by a different vector present in the host cell. In some methods, a library of test agents is encoded by a library of such vectors (e.g., a cDNA library). Such a library may be created by methods well known in the art (Sambrook et al. and Ausubel et al., supra), or obtained from a variety of commercial sources.

In addition to the foregoing cell based assay, the test agents may be screened by a non-cell based method. The method encompasses, for example, mobility shift DNA-binding assay, methylation and uracil interference assay, DNase and hydroxyl radical footprinting analysis, fluorescence polarization, and UV crosslinking or cross-linkers. A general overview is disclosed in Ausubel et al. (Ausubel et al., supra, chapter 12, DNA-Protein Interaction). One technique for isolating co-associating proteins including nucleic acid and DNA/RNA binding proteins includes the use of UV crosslinking or chemical cross-linkers, including cleavable cross-linkers dithiobis (succinimidylpropionate) and 3, 3′-dithiobis (sulfosuccinimidyl-propionate) (McLaughlin, Am. J. Hum. Genet., 59:561-569, 1996; Tang, Biochemistry, 35:8216-8225, 1996; Lingner, Proc. Natl. Acad. Sci. U.S.A., 93:10712, 1996; and Chodosh, Mol. Cell. Biol., 6:4723-4733, 1986).

Hereinafter, the first assay and the second assay will be described in detail.

The first assay is for screening agents that bind to EPRS or Snail1 or regulating the binding, and the present assay may be selectively employed according to the need of a person skilled in the art. Hereinafter, EPRS will be given as an example. Specifically, in the first assay, the binding of a test agent to EPRS may be assayed by various methods, such as labeled in vitro protein-protein binding assay, electrophoretic mobility shift assay, immunoassay for detecting protein binding, functional assay (phosphorylation assay, etc.), and the like (U.S. Pat. Nos. 4,366,241: 4,376,110; 4,517,288 and 4,837,168; and Bevan et al., Trends in Biotechnology, 13:115-122, 1995; Ecker et al., Bio/Technology, 13:351-360, 1995; and Hodgson, Bio/Technology, 10:973-980, 1992). The test agent may be identified by detecting a direct binding with EPRS, for example, co-immunoprecipitation with the EPRS polypeptide (or protein) by an antibody against EPRS. The test agent may also be identified by detecting a signal that can indicate the binding of EPRS and the test agent, e.g., fluorescence quenching.

Competition assays provide a suitable format for identifying test agents that specifically bind to EPRS. In such a format, test agents are screened in competition with a compound already known to bind to EPRS. The known binding compound may be a synthetic compound. It can also be an antibody that specifically recognizes EPRS, e.g., a monoclonal antibody against EPRS. If the test agent inhibits the binding between EPRS and the known compound, the test agent may bind to EPRS. Numerous types of competitive binding assays are known in the art, for example, solid phase direct or indirect radioimmunoassay (RIA), solid phase direct or indirect enzyme immunoassay (EIA), sandwich competition assay (Stahli et al., Methods in Enzymology, 9:242-2453, 1983); solid phase direct biotin-avidin EIA (Kirkland et al., J. Immunol., 137:3614-3619, 1986); solid phase direct labeled assay, solid phase direct labeled sandwich assay (Harlow and Lane, Antibodies, A laboratory Manual, Cold Spring Harbor Press, 1988); solid phase direct label RIA using ¹²⁵I (Morel et al., Mol. Immuno., 25(1); 7-15, 1988; solid phase direct biotin-avidin EIA (Cheung et al., Virology, 176:546-552, 1990); and direct labeled RIA (Moldenhauer et al., Sacnd. J. Immunol., 32:77-82, 1990). Typically, the above assays involve the use of purified polypeptide bound to a solid surface or cells bearing an unlabelled test agent and a labeled reference compound. Competitive inhibition is measured by determining the amount of label bound to the solid surface or cells in the presence of the test agent. Modulating agents to be identified by competition assay include agents that bind to the same epitope as the control compound and agents that bind to an adjacent epitope sufficiently proximal to the epitope bound by the control compound in order to allow steric hindrance to occur. Usually, when competitive inhibition is excessively present, the specific binding of the control group to a general target polypeptide will be inhibited by at least 50 or 75%.

The screening assay may be in an insoluble or soluble format. One example of the insoluble assays is to immobilize EPRS or a fragment thereof onto a solid phase matrix. Then, the solid phase matrix is contacted with test agents for a time period sufficient to allow the test agents to bind. After that, any unbound material is washed away from the solid phase matrix, and then the presence of the agent bound to the solid phase is identified. The method may further include a step of eluting the bound agent from the solid phase matrix, thereby isolating the agent. Alternatively, another method for immobilizing EPRS is to allow the test agents bind to the solid matrix and then add EPRS.

The soluble assay includes some of the combinatory libraries screening methods described above. Under the soluble assay format, neither the test agents nor EPRS binds to a solid support. The binding of EPRS or the fragment thereof to a test agent may be determined by, e.g., fluorescence of EPRS and/or the test agents. Fluorescence may be intrinsic or conferred by labeling a component with a fluorophor.

In some binding assays, EPRS, the test agent, or a third material (e.g., an antibody binding to EPRS) may be provided in a labeled state, in order to facilitate identification, detection and quantification of the polypeptide in a given condition. That is, EPRS, the test agent, or the third material may be provided by being covalently attached or linked to a detectable label or group, or cross-linkable group. These detectable groups include a detectable polypeptide group, e.g., an assayable enzyme or antibody epitope. Alternatively, the detectable group may be selected from other detectable groups or labels, such as radioactive isotopes (e.g., ¹²⁵I, ³²P, and ³⁵S) or a chemiluminescent or fluorescent group. Similarly, the detachable group may be a substrate, a cofactor, an inhibitor, or an affinity ligand.

In the first assay, the binding of EPRS and the test agent indicates that the test agent is an EPRS modulator. The binding also indicates that the agent can modulate biological activity of Snail, preferably Snail1. Therefore, the test agent binding to EPRS needs to be further tested for whether the test agent has ability to modulate activity of Snail1.

Alternatively, the test agent binding EPRS may be further examined in order to measure its activity on EPRS. The presence, nature, or extent of such activity may be tested by an activity assay. The activity assay may confirm that the binding of the test agent to EPRS actually has a modulatory activity on SPIK. More often, the activity assays may be independently employed to identify test agents that modulate activity of EPRS (i.e., without the first step of assaying the ability to bind to EPRS). Generally, the above methods include adding the test agent to a sample containing EPRS in the presence or absence of a different material or reagent necessary for testing biological activity of EPRS, and measuring a change in biological activity of EPRS. In addition to the assay for screening enzymes or other biological activity of EPRS, the activity assay includes in vitro screening and in vivo screening for the expression of EPRS or the change in the intracellular level.

The second assay stage means performing the screening method of one or more exemplary embodiments including steps (a) and (b) or steps (a) to (c). Once it is identified that the test agent binds to EPRS and/or modulates biological activity (including intracellular level) of EPRS in the first assay, the test agent may be further tested for whether it regulates the EMT pathway through Snail1, and further whether the test agent has the ability to prevent or treat the EMT-related (mediated) diseases. The regulation by the modulating agent is generally tested in the presence of EPRS. If the cell-based screening system is employed, EPRS may be expressed from an expression vector introduced into the host cell. Alternatively, EPRS may be inherently supplied by host cells.

Hereinafter, further exemplary embodiments will be described in detail.

However, the following examples are merely for illustrating the inventive concept, and are not intended to limit the scope of the present invention.

<Methods>

1. Cell Culture and Materials

Below mentioned cell lines of non-small cell lung cancer (NSCLC) and normal cell line were used in this experiment: adenocarcinoma (A549, HCC44), large-cell (H1299), embryonic kidney cell line HEK293T cell line.

HCC44 and H1299 NSCLC cell lines were maintained in RPMI and HEK293T cells were maintained in DMEM containing 10% fetal bovine serum with 1% antibiotics at 37° C. in a 5% CO₂ incubator. For transiently transfection, Fugene HD (Roche) and Lipofectamine 2000 (Invitrogen) were used and for siRNA transfection, Lipofectamine RNAiMAX (Inbitorgen) reagents were used, following the manufacturer's instruction. Nuclear and cytosol extracts were obtained using a commercial kit of nuclear fractionation (Active motif) and TGF-β1 (10 ng/ml) was purchased from R&D systems.

2. Plasmid and siRNA

cDNA encoding human EPRS (SEQ ID NO: 1) was purchased from Origene, and the EPRS cDNA represented by SEQ ID NO: 8, and each of cDNAs represented by SEQ ID NO: 9 and SEQ ID NO: 9, which encode ERS-WHEP domain (SEQ ID NO: 2) and WHEP-PRS domain (SEQ ID NO: 4), respectively, together with Strep tag (located at the N-terminal of a target protein) were subcloned into pEXPR-IBA-105(iba) plasmids. The thus constructed expression vectors pEXPR-2×IBA105-EPRS, pEXPR-IBA105-ERS, and pEXPR-IBA105-PRS were allowed to selectively transfect cells for the respective experiments. In addition, human Flag-tagged EPRS expression vector was purchased from Origene.

For the expression of Snail1, the pCMV-Tag2B N-terminal Flag-tagged Snail1 expression vector was purchased from Addgene. In addition, the Strep-tagged Snail expression vector (pEXPR-IBA105-Snail1) was constructed using pEXPR-IBA-105 vector in the same manner as described above. The pEXPR-IBA105-Snail1 expression vector expresses the Snail1 protein of SEQ ID NO: 15, including the DNA sequence represented by SEQ ID NO: 16.

As a control group, Strep-tag empty vector and Flag-tag empty vector were constructed by tagging only Strep or Flag to the pEXPR-IBA-105 vector. In addition, the HA-tagged ubiquitin expression vector was purchased from Addgene.

Human EPRS gene specific siRNA (si-EPRS) including a sense strand of SEQ ID NO: 23 and an antisense strand of SEQ ID NO: 24, and non-silencing control siRNA (si-control, Catalog Number: 4390843) were purchased from Invitrogen Silencer Select siRNAs products.

3. Cell Migration Assay

Cell migration assays with A549 cells were performed in Transwell chambers (8.0 μM pore, Costar). Fibronectin (10 μg/ml, BD Biosciences) was coated on the membrane and 700 μl serum-free RPMI was placed in the bottom chamber. After A549 cells were trypsinized and centrifuged at 1,000 rpm for 5 minutes, serum-free RPMI was added to the cells for suspension. Then, 1×10⁵ cells were dropped into 24-well Transwell chambers and incubated for 12 h at 37° C. in a CO₂ incubator. After washing the membrane twice with PBS, 70% methyl alcohol in PBS was used for cell fixation. PBS washing was performed three times afterwards and hematoxylin (Sigma) was used for cell staining After washing the membrane with distilled water several times, non-migrant cells from the top were removed by using cotton swab. Then, the membranes were mounted with Gel Mount (Biomeda). For counting migrant cells, three randomly picked places were magnified with microscopes in high-power fields (×10, and ×20). All samples were performed in triplicate.

4. Immunoprecipitation (IP)

HEK293T cells transiently co-transfected with Strep-tagged EPRS and Flag-tagged Snail were lysed with 20 mM Tris-HCl (pH 8.0) buffer containing 150 mM NaCl, 1% Triton X-100, 1 mM EDTA, 10% glycerol, and protease inhibitor (Calbiochem). To purify Strep-tagged protein, using MagStrep Type 2HC beads (IBA lifesciences) followed by manufacturer's instruction.

For the detection of endogenous EPRS interaction with Snail1 in nucleus and cytosol, the protein extracts were incubated with protein A agarose (Life technologies) for 30 minutes on ice, and then centrifuged to remove nonspecific IgG binding proteins. The pre-cleared supernatants were mixed with anti-EPRS antibody, incubated the mixture for 2 h at 4° C. with gentle agitation, added protein A agarose for 2 h, and centrifuged. After washing the precipitates with the cold lysis buffer 3-5 times, the precipitates were dissolved in the 2×SDS sample buffer and separated by SDS-PAGE.

5. Ubiquitination Assay

To examine the effects of EPRS on Snail1 ubiquitination, HA-tagged ubiquitin was co-transfected with Strep-Snail and Flag-Mock or Flag-EPRS in the presence of 50 μM MG132 for 4 h before harvest. The cell lysates were immunoprecipitated with streptavidin agarose (GE healthcare lifesciences). The beads were washed three times with the cold washing buffer, the precipitates were dissolved in the 2×SDS sample buffer and subjected to SDS-PAGE. Subsequently, Western blot analysis was performed with anti-HA antibody.

6. Western Blot Analysis

Cells were lysed with lysis buffer (20 mM Tris-HCl (pH 8.0) buffer containing 150 mM NaCl, 1% Triton X-100, 1 mM EDTA, 10% glycerol, and protease inhibitor) and collected in a 1.5 ml tube and incubated in 4° C. with gentle agitation for 15 minutes. Then, centrifuged at 15,000 rpm for 20 minutes in 4° C. and supernatants were collected and quantified by using Bradford reagent. After 5×SDS sample buffer was added, each sample were boiled in 100° C. for 5 minutes. Protein samples were separated by SDS-PAGE, transferred to nitrocellulose membrane, and immunoblotted with specific antibodies. The blots were then reacted with horseradish peroxidase-coupled anti-mouse and anti-rabbit (Pierce) IgG secondary antibody. Signals were developed with ECL-PLUS detection reagent (GE healthcare lifesciences).

The antibodies specific to the respective marker proteins, used in the present experiment, are as follows: StrepMAB classic-HRP (IBA), Monoclonal anti-FLAG M2-HRP (Sigma), anti-Snail1 (Cell signaling), anti-Slug (Cell signaling), anti-L13a (Cell signaling), anti-p-Smad2 (Cell signaling), anti-p-Smad3 (Cell signaling), anti-E-cadherin (Cell signaling), anti-N-cadherin (Cell signaling), anti-HDAC1 (Bio vision), anti-β-actin (SCBT), anti-Tubulin (Sigma), anti-HSP90 alpha/β (SCBT), anti-p84 (SCBT), and anti-HA (SCBT). The anti-EPRS antibody was prepared through a conventional procedure by which the EPRS protein represented by SEQ ID NO: 1 was injected into a New Zealand white rabbit to cause an immune response, thereby obtaining an antibody.

7. qRT-PCR Assay

A treatment group and a non-treatment group were prepared by treating A549 cells transfected with the strep-tag empty vector or strep-tagged EPRS expression vector with or without TGF-β1 (10 ng/ml) for 12 hours. After that, all RNAs were extracted using the GeneJET RNA Purification kit (Thermo Scientific). cDNA was synthesized using RNA 2 μg through the Maxima first strand cDNA synthesis kit for RT-qPCR (Thermo Scientific, #K1641) according to the manufacturer's protocol. qRT-PCR was carried out using the cDNA template, SYBR green Master MIX with ROX solution (Thermo Scientific), and 0.3 uM of forward and reverse primers listed in table 1 below, through the ABI 7500 instrument (life technologies). The qRT-PCR was conducted at 95° C. for denaturation and 60° C. for primer annealing/polymerase extension.

TABLE 1

| | | | SEQ |
|---|---|---|---|
| Target | Direction | Sequence | ID NO |
| EPRS | Forward | 5′-ATCCGCGTTAGAGCTGATTT-3′ | 17 |
| EPRS | Reverse | 5′-TAATGGGAACTCCCTTGAGC-3′ | 18 |
| SNAIL1 | Forward | 5′-CCTCCCTGTCAGATGAGGAC-3′ | 19 |
| SNAIL1 | Reverse | 5′-CCAGGCTGAGGTATTCCTTG-3′ | 20 |
| GAPDH | Forward | 5′-CAATGACCCCTTCATTGACC-3′ | 21 |
| GAPDH | Reverse | 5′-GACAAGCTTCCCGTTCTCAG-3′ | 22 |

8. Cell Invasion Assay

Cell invasion assay using A549 cells were conducted in the Matrigel invasion chamber manufactured by BD Co. According to the manufacturer's protocol, the matrigel was rehydrated for two hours and 750 μl of the serum-free RPMI was located in a lower chamber. The A549 cells were trypsinized, and then centrifuged at 1,000 rpm for 5 minutes. The serum-free RPMI was added to the cells to prepare a suspension. Then, 1×10⁵ cells were put in the upper chamber, and then incubated in a CO₂ incubator at 37° C. for 12 hours. The membrane was washed two times with PBS, and the cells were fixed using PBS containing 70% methyl alcohol. Then, the membrane was washed three times with PBS, and then the cells were stained with hematoxylin (Sigma). The membrane was washed several times with distilled water, and then immobilized cells above were removed using cotton swabs. Then, the membrane was mounted on the Gel Mount (Biomeda). For counting moving cells, three randomly selected regions were magnified through the microscope. All samples were repeatedly tested three times.

### Example 1

### Verification on Interaction Between Nucleus EPRS and Snail1

<1-1> Verification on Interaction at Endogenous Level of Cancer Cells

The interaction between EPRS and Snail1 were verified at the endogenous level of cytoplasm and nucleus. A549 cells were treated with TGF-β1 of different concentrations (0, 1, 10, 20 ng/ml) for 48 hours, fractionated into the cytoplasm and the nucleus, and then subjected endo-IP using protein A-agarose beads (Invitrogen) and EPRS antibody.

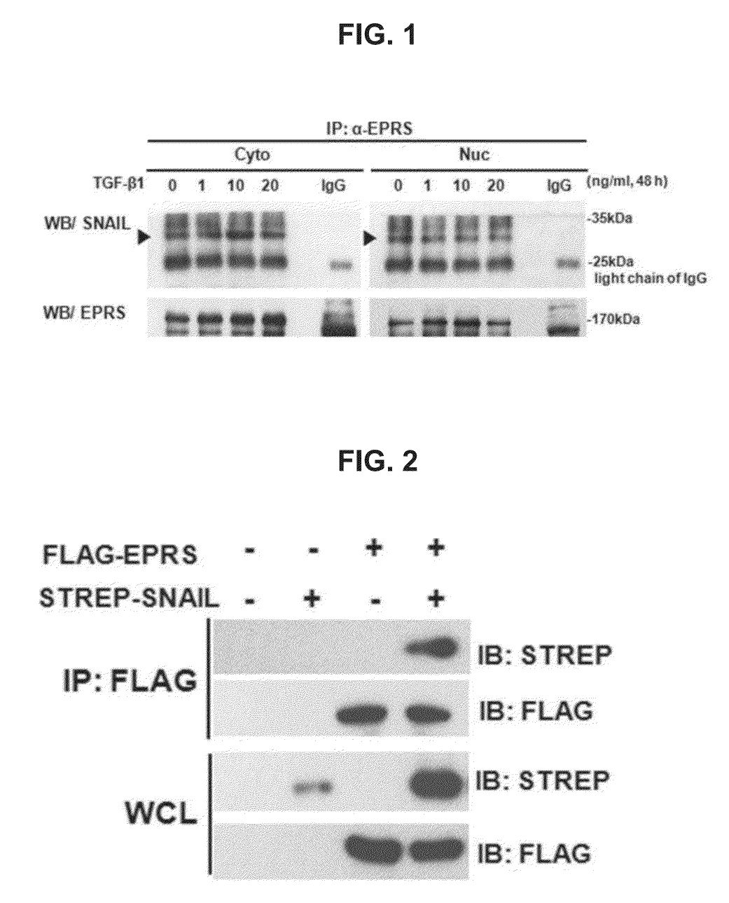

The sample subjected to IP was immunoblotted with Snail1 antibody, and as a result, it was confirmed that the higher the concentration of TGF-β1, the stronger the binding between EPRS and Snail1, as shown in FIG. 1.

<1-2> Verification on Interaction by Co-Transfection

The interaction between EPRS and Snail1 was verified through IP after co-transfection of the proteins. HEK293T cells were co-transfected with (1) FLAG-tag empty vector and STREP-tag empty vector, (2) FLAG-tag empty vector and STREP-tagged Snail1 expression vector, (3) FLAG-tagged EPRS expression vector and STREP-tag empty vector, (4) FLAG-tagged EPRS expression vector and STR EP-tagged Snail1 expression vector, that is, in a total of four types of conditions, and then respective proteins were overexpressed, and then IP was performed using the anti-FLAG M2 affinity gel (Sigma).

The samples subjected to FLAG IP under the four types of conditions were immunoblotted with the STREP antibody, and as a result, it was confirmed that the overexpressed EPRS and Snail1 react with each other, as shown in FIG. 2.

<1-3> Verification on Interaction in Snail1 Overexpression Condition

The interaction between EPRS and Snail1 was verified through IP after Snail1 overexpression. HEK293T cells were transfected with 1) STREP-tag empty vector and (2) STREP-tagged Snail1 expression vector, that is, in a total of two types of conditions, and then respective proteins were overexpressed, and then IP was performed using MagSTREP type 2HC beads (IBA).

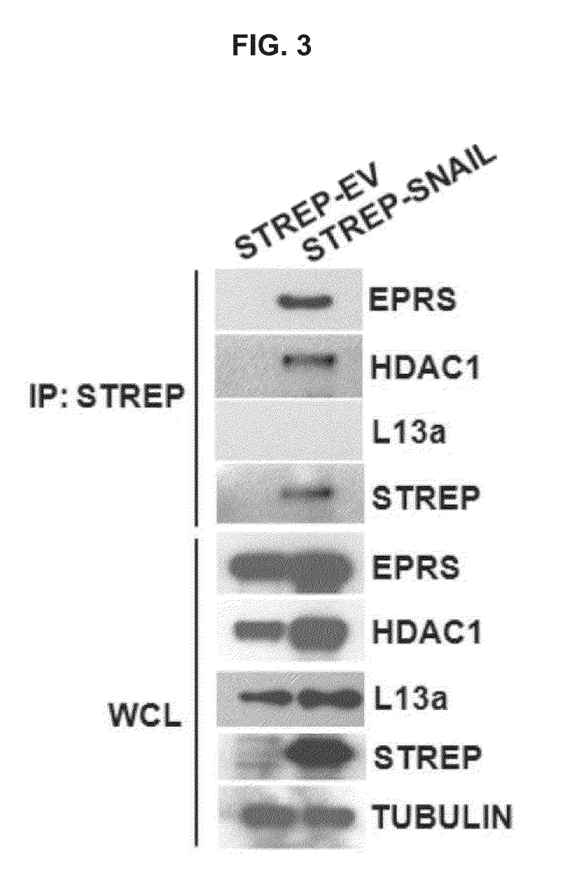

The samples subjected to STREP IP under the two types of conditions were immunoblotted with the EPRS antibody, and the results were shown in FIG. 3. The EPRS band was not observed in the empty vector overexpressed sample, and the EPRS band was observed in the Snail1 overexpressed sample. That is, the interaction between the overexpressed Snail1 and EPRS was verified. Here, in order to validate that the overexpressed Snail1 was well subjected to IP, HDAC1, which is known to interact with Snail1 was immunoblotted together. In order to verify whether components that are known to form the GAIT complex together with EPRS by IFN-r signaling to regulate translation (Mukhopadhyay, Jia et al. 2009) also interact with Snail1, L13a, which is one of such components, was immunoblotted. As a result, the interaction with Snail1 was not observed.

<1-4> Verification on Interaction Between EPRS Fragment and Snail1

EPRS was divided into ERS-WHEP domain (SEQ ID NO: 2), WHEP-PRS domain (SEQ ID NO: 4), and EPRS (SEQ ID NO: 1), and then the interaction with Snail1 and each domain was verified through IP after co-transfection. HEK293T cells were transfected with (1) STREP-tag empty vector and FLAG-tag empty vector, (2) STREP-tagged ERS-WHEP expression vector and FLAG-tagged Snail1 expression vector, (3) STREP-tagged WHEP-PRS expression vector and FLAG-tagged Snail1 expression vector, and (4) STREP-tagged EPRS expression vector and FLAG-tagged Snail1 vector, in a total of four types of conditions, and then respective proteins were overexpressed, and then IP was performed using MagSTREP type 2HC.

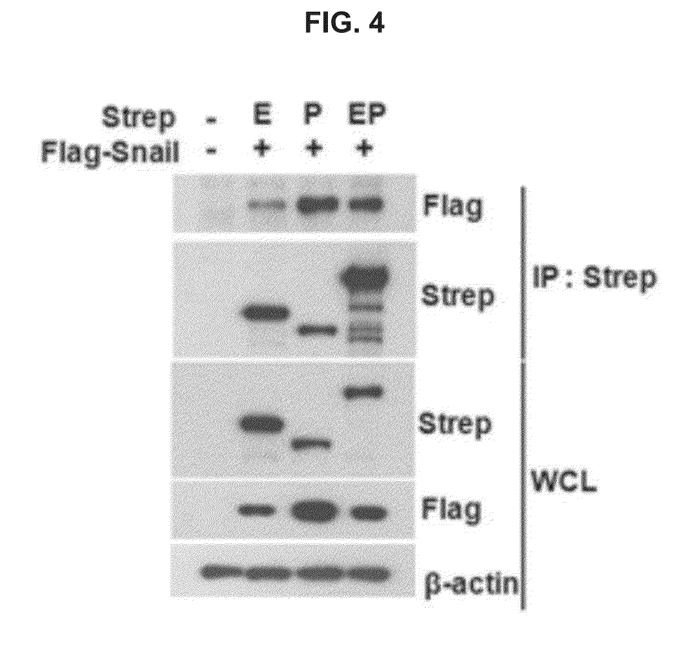

The samples subjected to STREP IP under the four types of conditions were immunoblotted with the FLAG antibody, and as a result, it was confirmed that the overexpressed ERS-WHEP, WHEP-PRS, and EPRS react with Snail1, as shown in FIG. 4. Of these, the interaction with Snail1 was observed to be stronger in WHEP-PRS rather than in ERS-WHEP and EPRS.

### Example 2

### Verification on Increase in Snail1 Stability of EPRS Through Physical Interaction

<2-1> Verification on Effect at Protein Level

For an experiment to verify how the EPRS overexpression affects the expression of Snail1, each type of A549, HCC44, and 293T cells were divided into a total of four types of experimental groups: (1) an experimental group untreated with TGF-β1 after overexpression of the empty vector, (2) an experimental group treated with TGF-β1 after overexpression of the empty vector, (3) an experimental group untreated with TGF-β1 after overexpression of EPRS, and (4) an experimental group treated with TGF-β1 after overexpression of EPRS. Here, the groups treated with TGF-β1 were prepared by treatment at a concentration of 10 ng/ml for 1 hour. The cells of the four experimental groups were lysed, and then immunoblotted with Snail1 antibody, respectively.

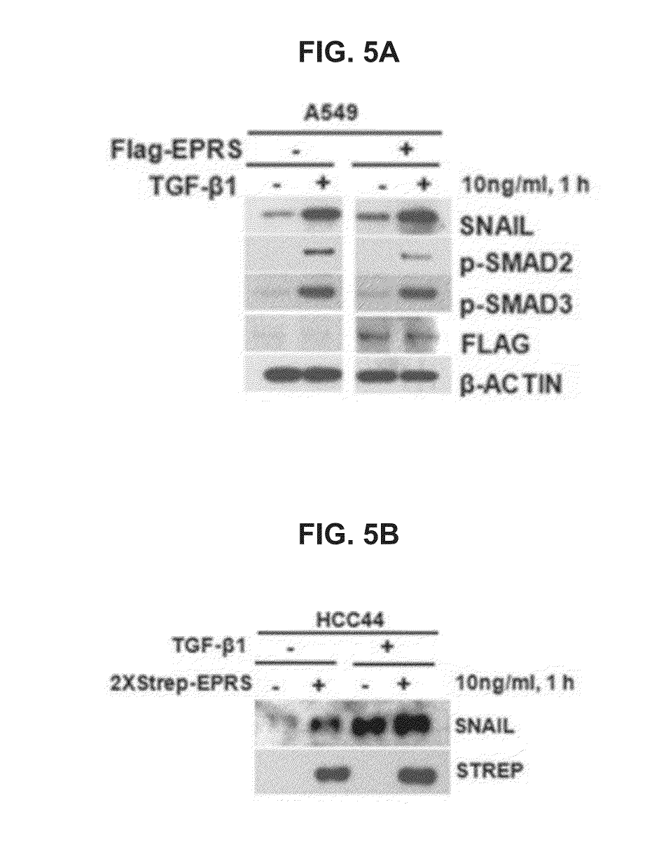

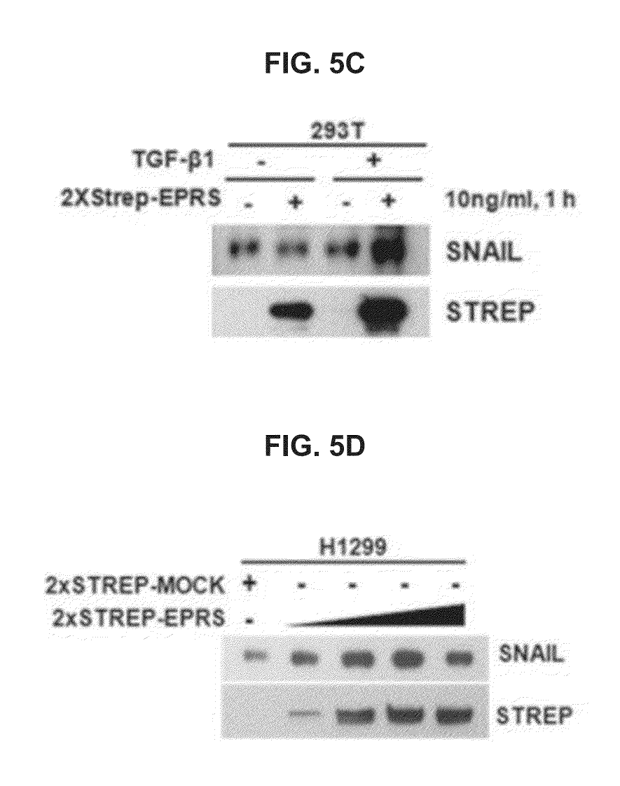

As a result, it was confirmed from FIGS. 5A, 5B, and 5C that, in all of A549, HCC44, 293T cell stains, the EPRS overexpression increased the Snail expression even without TGF-β1 treatment, and the TGF-β1 treatment grew the increase ratio, through the immunoblotting using Snail1 antibody.

Besides, it was confirmed from FIG. 5D that, in H1299 cells, as the expression level of EPRS gradually increased, the expression level of Snail1 also further increased through the immunoblotting using Snail1 antibody (in FIG. 5D, the sign “ [symbol] ” means that the expression level of the corresponding protein (that is, EPRS) increases in the cells).

<2-2> Verification at mRNA Expression Level

For an experiment to verify how the EPRS overexpression affects the change in the mRNA level of Snail1, A549 cells were divided into a total of four types of experimental groups: (1) an experimental group untreated with TGF-β1 after overexpression of the empty vector, (2) an experimental group treated with TGF-β1 after overexpression of the empty vector, (3) an experimental group untreated with TGF-β1 after overexpression of EPRS, and (4) an experimental group treated with TGF-β1 after overexpression of EPRS, and then qRT-PCR was conducted. Here, the groups treated with TGF-β1 were prepared by treatment at a concentration of 10 ng/ml for 12 hours.

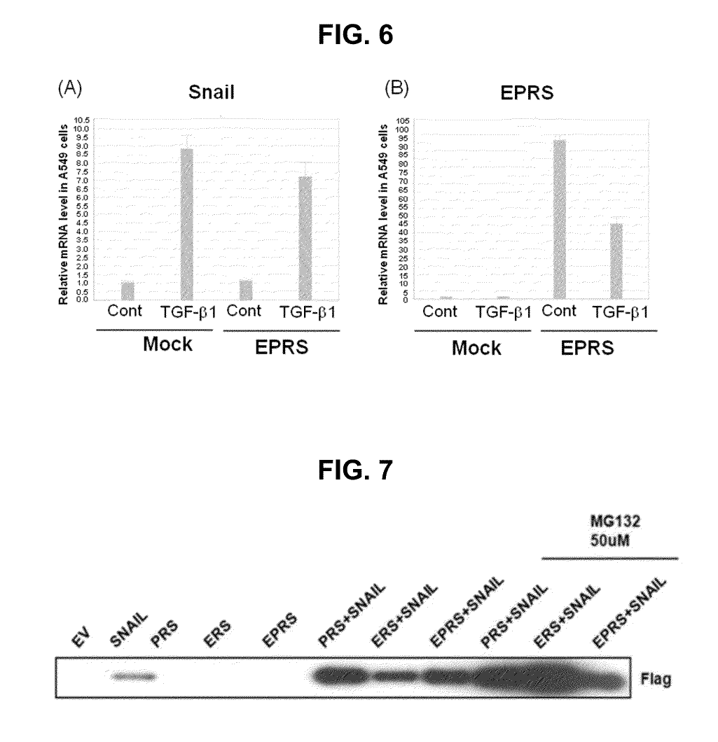

As a result, as shown in FIG. 6, the EPRS overexpression did not change the relative mRNA level of Snail1. Thus, it may be predicted that EPRS may interact with Snail in a manner except for regulating the mRNA of Snail1.

<2-3> Verification on Increase in Snail1 Stability of EPRS

For an experiment to verify whether EPRS increases Snail1 stability, through the treatment with MG132 as a proteasomal inhibitor, HEK293T cells were divided into a total of eleven types of experimental groups: experimental groups co-transinfected with (1) STREP-tag empty vector and FLAG-tag empty vector, (2) STREP-tag empty vector and FLAG-tagged Snail1, (3) STREP-tagged WHEP-PRS and FLAG-tag empty vector, (4) STREP-tagged ERS-WHEP and FLAG-tag empty vector, (5) STREP-tagged EPRS and FLAG-tag empty vector, (6), (7), and (8) FLAG-tag Snail1 instead of FLAG-tag empty vector in (3), (4), and (5) above, and (9), (10), and (11) further treated with 50 μM of MG132 for 6 hours in (6), (7), and (8) above. The cells for the above experimental groups were lysed, and immunoblotted with anti-Flag antibody, respectively.

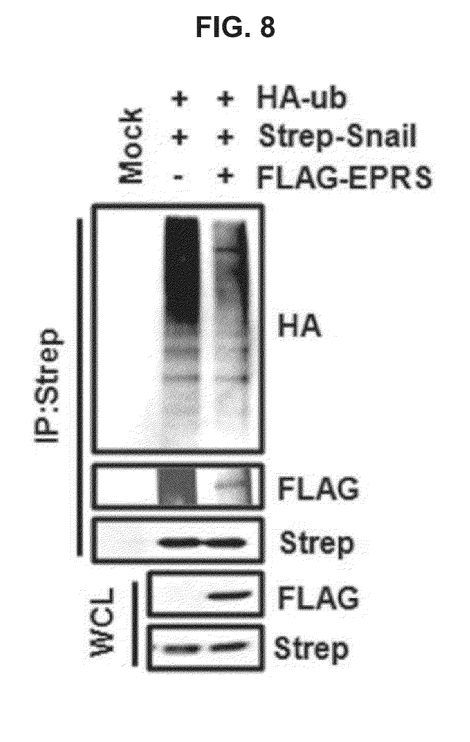

As a result, as shown in FIG. 7, it was observed that the protein expression level of Snail1 increased more when overexpressed together with EPRS through co-transfection than when overexpressed alone, and the WHEP-PRS fragment rather than ERS-WHEP fragment in the EPRS more contributed to the Snail1 stability. This finding is consistent with the results showing that the EPRS overexpression had an effect of inhibiting poly-ubiquitination of Snail1, through the ubiquitination assay in FIG. 8.

<2-4> Verification on Increase in Snail1 Stability Through EPRS Overexpression

For an experiment to verify whether the EPRS overexpression regulates the Snail1 stability through the ubiquitination inhibitory action, HA-tagged ubiquitin A549 cells were co-transfected with (1) FLAG-tag empty vector and STREP-tag Snail1, and (2) FLAG-tagged EPRS and STREP-tag Snail1, that is, in a total of two types of conditions, and treated with 50 μM of MG-132 for 6 hours before harvest, and then IP was conducted using Streptavidin beads.

The samples subjected to IP were immunoblotted with HA antibody. As a result, as shown in FIG. 8, the ubiquitination was reduced in the EPRS overexpressed experimental group. It is well known that Snail1 degradation is regulated by its ubiquitination (Zhou, Deng et al. 2004), and thus it can be predicted that the EPRS overexpression reduces the ubiquitination of Snail1, thereby increasing the Snail1 stability.

### Example 3

### Verification on EMT Progress Inhibiting Effect by Inhibiting Binding Between EPRS and Snail

<3-1> Verification on Effect of EPRS Knock-Down on Snail1 and EMT Progress

It was verified how the reduced EPRS affects Snail1 and EMT progress, through EPRS knock-down experiment using siRNA. For each experimental group, A459 cells were treated with si-control or si-EPRS, and treated with or without 10 ng/ml of TGF-β1 for 1 hour or 48 hours. The changes of EMT-related markers (p-SMAD3, SMAD2/3, SLUG EPRS, E-cadherin, and N-cadherin) including Snail1 were observed by immunoblotting for the cytoplasm and the nucleus. HSP90α/β and p84 were used as loading controls for confirming the protein expression levels in the cytoplasm and nucleus each.

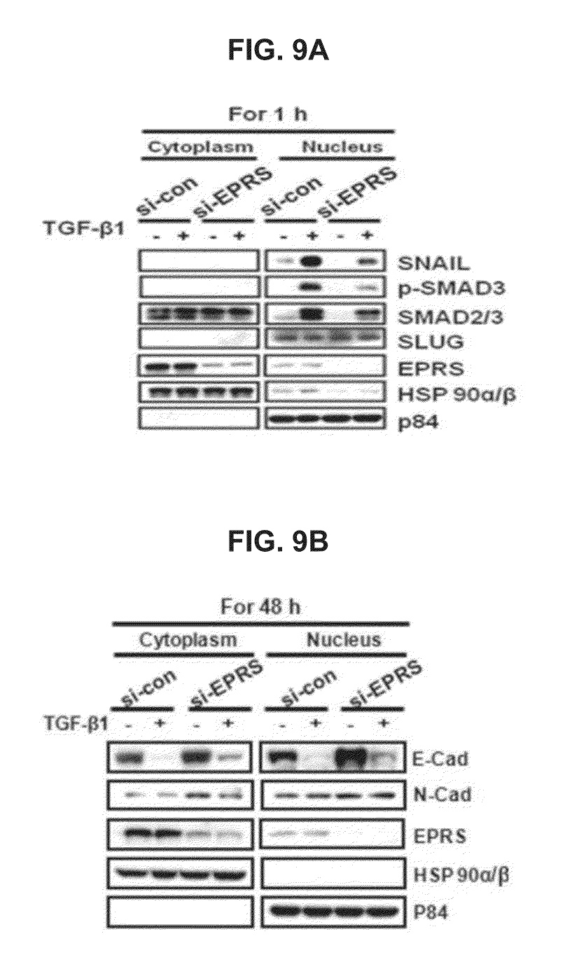

As a result, as shown in FIGS. 9A and 9B, at the time of EPRS knock-down, the Snail1 and p-SMAD3 was reduced and the E-cadherin was increased. This indicates that the EMT progress was suppressed.

<3-2> Verification on Effect of EPRS Knockdown on Cell Migration

For an experiment to verify how the reduced EPRS affects cell migration, cell migration was observed for a total of four experimental groups in which A549 cells were treated (1) without TGF-β1 after si-control treatment, (2) with TGF-β1 after si-control treatment, (3) without TGF-β1 after si-EPRS treatment, and (4) with TGF-β1 after si-EPRS treatment. Here, the TGF-β1 treatment groups were treated at a concentration of 10 ng/ml for 12 hours.

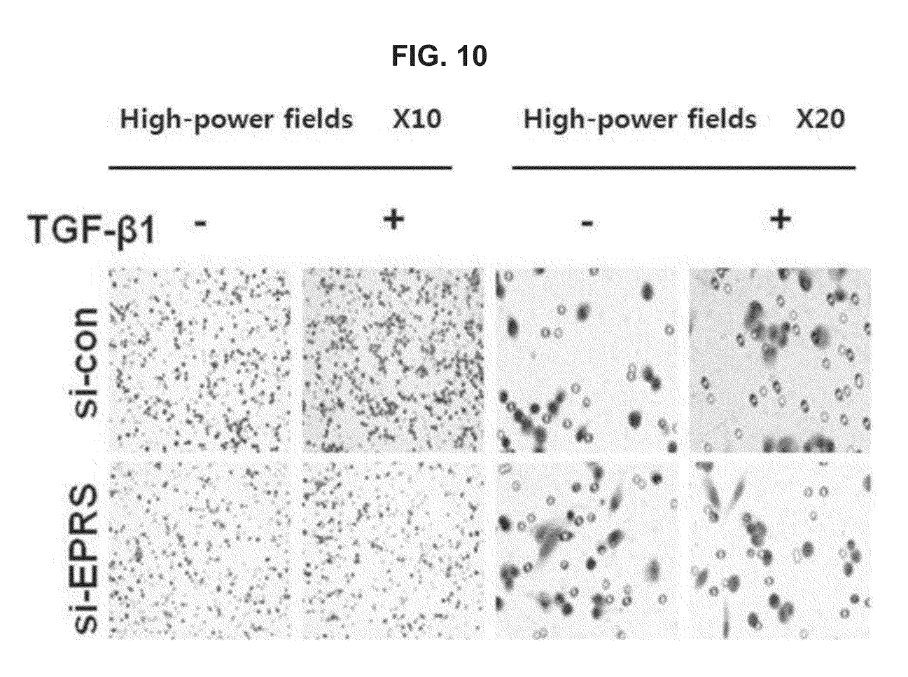

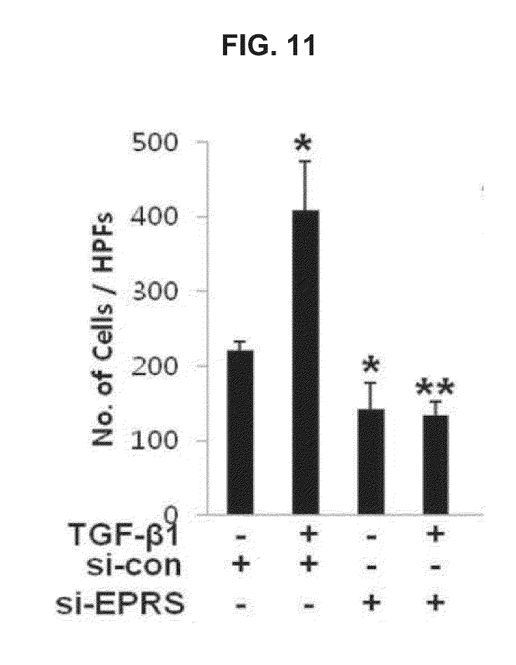

As a result, as shown in FIGS. 10 and 11, the cell migration was reduced at the time of EPRS knock down. Here, the cell migration was reduced in all of the groups treated with and without TGF-β1.

<3-3> Verification on Effect of EPRS Knock-Down on Cell Invasion

For an experiment to verify how the reduced EPRS affects cell invasion, cell invasion was observed for a total of four types of experimental groups in which A549 cells were treated (1) without TGF-β1 after si-control treatment, (2) with TGF-β1 after si-control treatment, (3) without TGF-β1 after si-EPRS treatment, and (4) with TGF-β1 after si-EPRS treatment. Here, the groups treated with TGF-β1 were prepared by treatment at a concentration of 10 ng/ml for 12 hours.

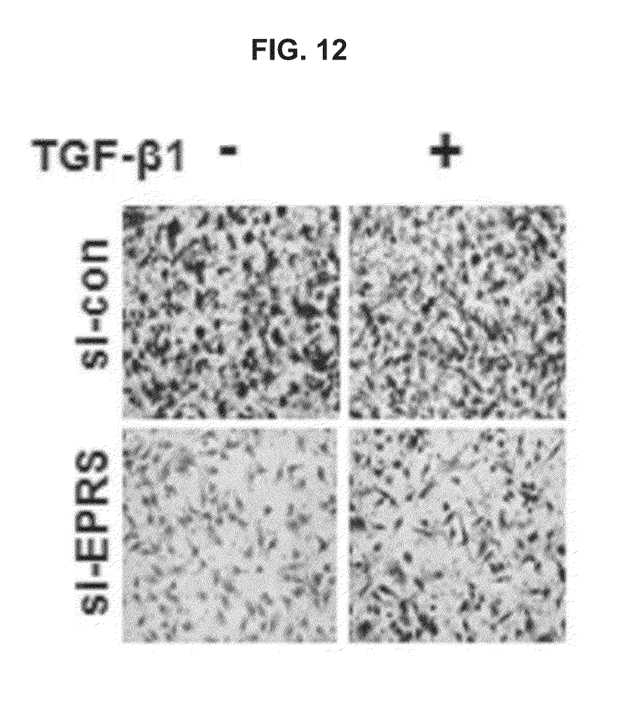

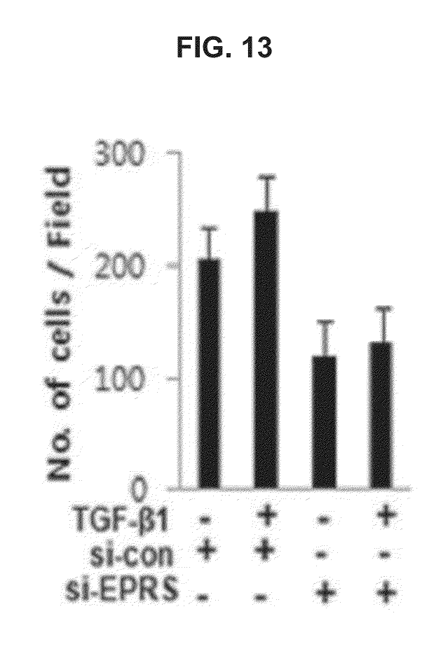

As a result, as shown in FIGS. 12 and 13, the cell invasion as well as the cell migration verified in examples <3-2> were reduced at the time of EPRS knock-down. Here, the cell invasion was reduced in all the groups treated with and without TGF-β1.

The increases in cell migration and invasion are main characteristics of EMT progress cells (especially, cancer cells), and the above experimental results confirmed that the inhibition of the interaction between EPRS and Snail1 can suppress the EMT progress. This suggests a main target in the development of therapeutic agents for diseases that have been known to be relevant to EMT, such as cancer (metastasis) and fibrosis.

Although certain exemplary embodiments and implementations have been described herein, other embodiments and modifications will be apparent from this description. Accordingly, the inventive concept is not limited to such embodiments, but rather to the broader scope of the presented claims and various obvious modifications and equivalent arrangements.

What is claimed is:

1. A method for screening an epithelial-mesenchymal transition (EMT) inhibitor, the method comprising:
contacting glutamyl-prolyl-tRNA synthetase (EPRS) having an amino acid sequence, Snail1 protein, and a test agent, the amino acid sequence being selected from the group consisting of SEQ ID NOs: 2 to 6; and
measuring a change in a binding level between the EPRS and the Snail1 protein.

2. The method according to claim 1, wherein the epithelial-mesenchymal transition inhibitor is an agent for preventing or treating diseases or symptoms selected from the group consisting of cancer metastasis, fibrotic disease, angiogenesis, diabetic renal nephropathy, allograft dysfunction, cataracts, and defects in cardiac valve formation.

3. The method according to claim 2, wherein a cancer for the cancer metastasis is selected from the group consisting of non-small cell lung cancer, small cell lung cancer, melanoma, leukemia, colon cancer, liver cancer, gastric cancer, esophageal cancer, pancreatic cancer, gallbladder cancer, kidney cancer, bladder cancer, prostate cancer, testicular cancer, cervical cancer, endometrial cancer, choriocarcinoma, ovarian cancer, breast cancer, thyroid cancer, brain cancer, head and neck cancer, skin cancer, lymphoma, aplastic anemia, bile duct cancer, oral cancer, peritoneal cancer, small intestine cancer, and eye tumor.

4. The method according to claim 2, wherein the fibrotic disease is selected from the group consisting of renal fibrosis, hepatic fibrosis, pulmonary fibrosis, skin fibrosis, cardiac fibrosis, joint fibrosis, nerve fibrosis, muscular fibrosis, and peritoneal fibrosis.

5. The method according to claim 1, wherein the glutamyl-prolyl-tRNA synthetase has an amino acid sequence selected from the group consisting of SEQ ID NO: 1 and SEQ ID NOs: 25 to 123.

6. The method according to claim 1, wherein the Snail1 has an amino acid sequence selected from the group consisting of SEQ ID NO: 15 and SEQ ID NOs: 124 to 203

7. The method according to claim 1, wherein the test agent is at least one agent selected from the group consisting of protein, polypeptide, small organic molecule, polysaccharide, and polynucleotide.

8. The method according to claim 1, further comprising:
contacting the test agent, which has changed the binding level between the EPRS and the Snail1 protein, with cells expressing Snail1, together with TGF-β1, and then verifying an EMT inhibitory effect in the cells.

9. The method according to claim 8, wherein the cells expressing Snail1 are selected from the group consisting of normal epithelial cells, non-small cell lung cancer cells, small cell lung cancer cells, melanoma cells, leukemia cells, colon cancer cells, liver cancer cells, gastric cancer cells, esophageal cancer cells, pancreatic cancer cells, gallbladder cancer cells, kidney cancer cells, bladder cancer cells, prostate cancer cells, testicular cancer cells, cervical cancer cells, endometrial cancer cells, choriocarcinoma cells, ovarian cancer cells, breast cancer cells, thyroid cancer cells, brain cancer cells, head and neck cancer cells, skin cancer cells, lymphoma cells, aplastic anemia cells, bile duct cancer cells, oral cancer cells, peritoneal cancer cells, small intestine cancer cells, eye tumor cancer cells, renal fibrosis cells, hepatic fibrosis cells, pulmonary fibrosis cells, skin fibrosis cells, cardiac fibrosis cells, joint fibrosis cells, nerve fibrosis cells, muscular fibrosis cells, and peritoneal fibrosis cells.

10. The method according to claim 1, wherein the EPRS is full-length EPRS or a fragment of EPRS.
